(19) United States
(12) Patent Application Publication
(10) Pub. No.: US 2023/0201086 A1
(43) Pub. Date: Jun. 29, 2023

(54) CELLULOSE ACETATE PARTICLES, COSMETIC COMPOSITION, AND METHOD FOR PRODUCING CELLULOSE ACETATE PARTICLES

(71) Applicant: Daicel Corporation, Osaka (JP)

(72) Inventors: Keiko Kobayashi, Tokyo (JP); Masaya Omura, Tokyo (JP)

(73) Assignee: Daicel Corporation, Osaka-shi, Osaka (JP)

(21) Appl. No.: 18/115,215

(22) Filed: Feb. 28, 2023

Related U.S. Application Data
(63) Continuation-in-part of application No. 17/926,426, filed on Nov. 18, 2022.
Application No. 17/926,426 is a continuation-in-part of application No. 18/010,591, filed on Jan. 1, 0001.
Application No. 18/010,591 is a continuation-in-part of application No. 16/616,160, filed on Nov. 22, 2019, now Pat. No. 11,628,134.
Application No. 16/616,160 is a continuation-in-part of application No. 17/435,128, filed on Aug. 31, 2021.

(30) Foreign Application Priority Data
Feb. 7, 2018 (JP) 2018-020422
May 23, 2018 (JP) 2018-099033
May 20, 2020 (JP) 2020-088329
Jul. 13, 2020 (JP) 2020-120253

(51) Int. Cl. A61K 8/02; A61K 8/73; A61Q 19/00

(52) U.S. Cl. CPC A61K 8/025; A61K 8/731; A61Q 19/00; A61K 2800/654; A61K 2800/412; A61K 2800/413; A61K 2800/10

(57) ABSTRACT

The present disclosure relates to a composition having cellulose acetate particles having a sphericity from 0.7 to 1.0 and a surface smoothness from 80% to 100% or less, and a method of manufacturing and use thereof.

The instant application is a Continuation-In-Part application of U.S. patent application Ser. No. 16/616,160 (U.S. Patent Application Publication No. 2020/0179261) filed Nov. 22, 2019, which is a national stage application of PCT/JP2019/004230 filed Feb. 6, 2019, claiming priority to Japanese Application No. 2018-099033 filed May 23, 2018 and Japanese Application No. 2018-020422 filed Feb. 7, 2018, the contents of each of which are incorporated herein by reference. The instant application is also a Continuation-In-Part application of U.S. patent application Ser. No. 17/435,128 (U.S. Patent Application Publication No. 2022/0142900) filed Aug. 31, 2021, which is a national stage application of PCT/JP2019/011161 filed Mar. 18, 2019, the contents of each of which are incorporated herein by reference. The instant application is also a Continuation-In-Part application of U.S. patent application Ser. No. 17/926,426 filed Nov. 22, 2019, which is a national stage application of PCT/JP2021/018428 filed May 14, 2021, claiming priority to Japanese Application No. 2020-088329 filed May 20, 2020, the contents of each of which are incorporated herein by reference. The instant application is also a Continuation-In-Part application of U.S. patent application Ser. No. 18/010,591 filed Nov. 22, 2019, which is a national stage application of PCT/JP2021/025809 filed Jul. 8, 2021, claiming priority to Japanese Application No. 2020-120253 filed Jul. 13, 2020, the contents of each of which are incorporated herein by reference.

### BACKGROUND ART

Cellulose acetate is a typical biodegradable polymer. Cellulose acetate is excellent in that it can be obtained from natural materials, such as wood and cotton, which do not conflict with food and feed. Thus, it would be beneficial if fine synthetic polymer particles could be replaced with cellulose acetate fine particles. However, polymers to which a method of producing fine synthetic polymer particles is applicable are limited, and it is difficult to apply the method to production of fine cellulose acetate particles.

### SUMMARY

The present specification relates to cellulose acetate particles having an average particle size of 80 nm or more and 100 μm or less, a sphericity of 0.7 or more and 1.0 or less, and a surface smoothness of 80% or more and 100% or less. In some embodiments, the cellulose acetate has a total degree of acetyl substitution of 0.7 or more and 2.9 or less. In the cellulose acetate particles, the total degree of acetyl substitution of the cellulose acetate may be 2.0 or more and less than 2.6. The cellulose acetate particles may contain a plasticizer, and a content of the plasticizer may be 2% by weight or more and 40% by weight or less based on a weight of the cellulose acetate particles. In the cellulose acetate particles, the plasticizer may be at least one or more selected from the group consisting of a citric acid-based plasticizer, a glycerin ester-based plasticizer, an adipic acid-based plasticizer, and a phthalic acid-based plasticizer. The present specification relates to a cosmetic composition containing the cellulose acetate particles. The present specification relates to a method of producing cellulose acetate particles, the method including mixing cellulose acetate with a plasticizer to obtain cellulose acetate impregnated with the plasticizer, kneading cellulose acetate impregnated with the plasticizer and a water-soluble polymer at 200° C. or more and 280° C. or less to obtain a dispersion having the cellulose acetate impregnated with the plasticizer as a dispersoid, and removing the water-soluble polymer from the dispersion. In the method of producing cellulose acetate particles, the mixing may be performed by mixing the cellulose acetate and the plasticizer in a temperature range of 20° C. or more and less than 200° C. and then melt-kneading. In the method of producing cellulose acetate particles, the plasticizer may be at least one or more selected from the group consisting of a citric acid-based plasticizer, a glycerin ester-based plasticizer, an adipic acid-based plasticizer, and a phthalic acid-based plasticizer. In the method of producing cellulose acetate particles, the plasticizer may be at least one or more selected from the group consisting of triethyl citrate, acetyl triethyl citrate, acetyl tributyl citrate, triacetin, and diisononyl adipate. In the method of producing cellulose acetate particles, the plasticizer may be at least one or more selected from the group consisting of acetyl triethyl citrate, triacetin, diacetin, and diethyl phthalate. In the method of producing cellulose acetate particles, the water-soluble polymer may be polyvinyl alcohol or thermoplastic starch.

The present specification also relates to particles containing cellulose acetate, in which the particles have an average particle size of not less than 80 nm and not greater than 100 μm, a sphericity of not less than 0.7 and not greater than 1.0, a degree of surface smoothness of not less than 80% and not greater than 100%, and a surface contact angle with water of not less than 100°. In some embodiments, a total degree of acetyl substitution of the cellulose acetate is not less than 0.7 and not greater than 2.9. In the particles, the surface contact angle with water may be not less than 120°. In the particles, the total degree of acetyl substitution of the cellulose acetate may be not less than 2.0 and less than 2.6. In the particles, the particles may contain a plasticizer, and a content of the plasticizer may be not greater than 1 wt. % relative to a weight of the particles. In the particles, the plasticizer may be at least one or more selected from the group consisting of a citrate-based plasticizer, a glycerin ester-based plasticizer, an adipate-based plasticizer, and a phthalate-based plasticizer. In the particles, the glycerin ester-based plasticizer may be triacetin. The present specification also relates to a cosmetic composition containing particles. The present specification also relates to a method for producing the particles, the method including surface-treating cellulose acetate particles with a lipophilicity-imparting agent, in which the cellulose acetate particles have an average particle size of not less than 80 nm and not greater than 100 μm, a sphericity of not less than 0.7 and not greater than 1.0, and a degree of surface smoothness of not less than 80% and not greater than 100%; and a total degree of acetyl substitution of the cellulose acetate is not less than 0.7 and not greater than 2.9. In the method for producing the particles, the lipophilicity-imparting agent may contain a silicone-based component. In the method for producing the particles, the surface treatment may be a surface treatment by a wet treatment method.

The present specification also relates to an emulsifiable preparation including: one or more aqueous components selected from the group consisting of water and alcohol; an oily component; and microparticles of a polymer compound, in which the microparticles contain cellulose acetate as the polymer compound, and the microparticles has an average particle size of 2 to 10 μm. The alcohol may contain a polyhydric alcohol. The amount of the polyhydric alcohol may be 20 wt. % or greater relative to the total amount of the alcohol. The emulsifiable preparation may further contain a thickener. The emulsifiable preparation may further contain a surfactant. The present disclosure provides an aqueous cosmetic, a food or beverage, or a pharmaceutical composition, each of which includes the emulsifiable preparation described above.

The present specification also relates to cellulose acetate particles, in which the cellulose acetate particles have an average particle size of 80 nm or greater and 100 μm or less, a sphericity of 0.7 or greater and 1.0 or less, and a relative specific surface area of 3.0 or greater and 20 or less. In some embodiments, a total degree of acetyl substitution of the cellulose acetate is 0.7 or greater and 3.0 or less. The cellulose acetate particles may have a degree of surface smoothness of 10% or greater and 95% or less. The cellulose acetate particles may have a bulk specific gravity of 0.2 or greater and 0.7 or less. In the cellulose acetate particles, the oil absorption using linseed oil may be 60 ml or greater per 100 g of the cellulose acetate particles. In the cellulose acetate particles, the cellulose acetate may have a total degree of acetyl substitution of 1.6 or greater and less than 2.9. The cellulose acetate particles may have a relative specific surface area of 10 or greater and 20 or less. The cellulose acetate particles contain a plasticizer, and a content of the plasticizer is 2 parts by weight or greater and 67 parts by weight or less relative to 100 parts by weight of the cellulose acetate. In the cellulose acetate particles, the plasticizer may contain at least one selected from the group consisting of a citrate-based plasticizer, a glycerin ester-based plasticizer, and a phthalate-based plasticizer. The present specification also relates to a cosmetic composition containing the cellulose acetate particles. The present specification also relates to a method for producing cellulose acetate particles including: mixing cellulose acetate, a plasticizer, a first thermoplastic polymer, and a second thermoplastic polymer, and forming a mixture of cellulose acetate containing the plasticizer, the first thermoplastic polymer, and the second thermoplastic polymer; melt-kneading the mixture at 200° C. or higher and 280° C. or lower; and removing the first thermoplastic polymer and the second thermoplastic polymer from the melt-kneaded mixture, in which when SPa represents an SP value of the cellulose acetate, SPb represents an SP value of the first thermoplastic polymer, and SPc represents an SP value of the second thermoplastic polymer, SPa, SPb, and SPc satisfy the following relationship: 0.1≤|SPc−SPa|/|SPb−SPa|≤0.9. In the method for producing cellulose acetate particles, the mixing may be performed by mixing the cellulose acetate, the plasticizer, the first thermoplastic polymer, and the second thermoplastic polymer at 20° C. or higher and lower than 200° C., and then melt-kneading the mixture. In the method for producing cellulose acetate particles, the first thermoplastic polymer may be a water-soluble polymer. In the method for producing cellulose acetate particles, the second thermoplastic polymer may be a water-soluble polymer. In the method for producing cellulose acetate particles, the plasticizer may contain at least one selected from the group consisting of a citrate-based plasticizer, a glycerin ester-based plasticizer, an adipate-based plasticizer, and a phthalate-based plasticizer. In the method for producing cellulose acetate particles, the plasticizer may contain at least one selected from the group consisting of triethyl citrate, acetyl triethyl citrate, acetyl tributyl citrate, triacetin, diacetin, diisononyl adipate, and diethyl phthalate. In the method for producing cellulose acetate particles, the plasticizer may contain at least one selected from the group consisting of acetyl triethyl citrate, triacetin, diacetin, and diethyl phthalate. In the method for producing cellulose acetate particles, the plasticizer may contain at least one selected from the group consisting of acetyl triethyl citrate and triacetin. In the method for producing cellulose acetate particles, the first thermoplastic polymer may contain at least one selected from the group consisting of polyvinyl alcohol and thermoplastic starch. In the method for producing cellulose acetate particles, the second thermoplastic polymer may be polyethylene glycol.

### BRIEF DESCRIPTION OF DRAWINGS

FIG. 1 is a scanning electron microscope (SEM) image (magnification: 3000 times) of cellulose acetate particles of Example A-1.

FIG. 2 is an SEM image (magnification: 5000 times) of cellulose acetate particles of Example A-1.

FIG. 3 is an SEM image (magnification: 6000 times) of cellulose acetate particles of Example A-1, and is a view for explaining a method for evaluating a degree of surface smoothness (%).

FIG. 4 is a binarized image of FIG. 3, and is a view for explaining the method for evaluating a degree of surface smoothness (%).

FIG. 5 is an SEM image (magnification: 3000 times) of cellulose acetate particles of Example A-12.

FIG. 6 is an SEM image (magnification: 5000 times) of cellulose acetate particles of Example A-12.

FIG. 7 is an SEM image (magnification: 3000 times) of cellulose acetate particles of Comparative Example A-1.

FIG. 8 is an SEM image (magnification: 5000 times) of cellulose acetate particles of Comparative Example A-1.

### DESCRIPTION

### Cellulose Acetate Particles

The cellulose acetate particles of the present disclosure are cellulose acetate particles, in which the cellulose acetate particles have an average particle size of 80 nm or greater and 100 μm or less, a sphericity of 0.7 or greater and 1.0 or less, and a relative specific surface area of 3.0 or greater and 20 or less; and a total degree of acetyl substitution of the cellulose acetate is 0.7 or greater and 3.0 or less.

Since the average particle size of the cellulose acetate particles of the present disclosure is 80 nm or greater and 100 μm or less, the average particle size may be 100 nm or greater, 1 μm or greater, 2 μm or greater, or 4 μm or greater. In addition, the average particle size may be 80 μm or less, 40 μm or less, 20 μm or less, or 14 μm or less. The particles with too large an average particle size are poor in tactile sensation. Alternatively, the particles with too small average particle size may be difficult to produce. Here, the tactile sensation includes, for example, skin feel and tactile sensation of a cosmetic composition containing the particles, in addition to tactile sensation in directly touching the cellulose acetate particles.

The average particle size can be measured using dynamic light scattering. The average particle size (such as in nm and μm) herein refers to the value of the particle size corresponding to 50% of the integrated scattering intensity in this particle size distribution.

The coefficient of variation of the particle size of the cellulose acetate particles of the present disclosure may be 0% or greater and 60% or less, or may be 2% or greater and 50% or less.

The coefficient of variation (%) of the particle size can be calculated by an equation: (standard deviation of particle size)/(average particle size)×100.

Since the sphericity of the cellulose acetate particles of the present disclosure is 0.7 or greater and 1.0 or less, the sphericity is preferably 0.8 or greater and 1.0 or less, and more preferably 0.9 or greater and 1.0 or less. The particles with a sphericity of less than 0.7 are poor in tactile sensation, and, for example, a cosmetic composition containing such particles leads to a reduction in skin feel.

The sphericity can be measured by the following method. The average value of the minor axis length/major axis length ratios of particles selected from an image of the particles observed with a scanning electron microscope (SEM) is defined as the sphericity. The closer to 1 the sphericity is, the closer to the true sphere the particles can be determined to be.

The cellulose acetate particles of the present disclosure have a relative specific surface area (may be hereinafter referred to as RSSA) of 3.0 or greater and 20 or less. The relative specific surface area is preferably 5.0 or greater, more preferably 7.0 or greater, still more preferably 8.5 or greater, yet still more preferably 9.0 or greater, and particularly preferably 10 or greater. Further, the relative specific surface area may be 18 or less. When the relative specific surface area is less than 3.0, truly spherical particles having a smooth surface are formed, the particles having too few or no pore portions. The particles are hardly deformed by an external force applied thereto, and the tactile sensation (particularly, softness) is poor. When the sphericity is more than 20, it is difficult for the particles to maintain high sphericity, and the particles are poor in tactile sensation.

The relative specific surface area (RSSA) is defined by the following relationship: RSSA=specific surface area measurement value/theoretical specific surface area

The theoretical specific surface area is a specific surface area calculated from the measurement results of the particle size distribution, assuming that the particle is a truly spherical particle having a smooth surface, and is defined as follows.

Theoretical specific surface area=(1/d)Σ(Pι*Sι/Vι)

d: true specific gravity (constant at 1350 (kg/m³))

Pι: distribution (volume fraction)

Sι; surface area (m²) of one cellulose acetate particle, i.e., a surface area (4/3)*π*(Lι/2)³ of a spherical cellulose acetate particle having a diameter Lι (m)

Vι: volume (m³) of one cellulose acetate particle, i.e., a volume 4π*(Lι/2)² of a spherical cellulose acetate particle having a diameter Lι (m)

The specific surface area measurement value is determined according to the BET specific surface area measurement by nitrogen adsorption.

The degree of surface smoothness of the cellulose acetate particles of the present disclosure is preferably 10% or greater and 95% or less, more preferably 50% or greater and 92% or less, and still more preferably 75% or greater and 90% or less. In the case of the particles with too small a degree of surface smoothness, the particles have too many portions (pore portions) corresponding to recesses. When the surface smoothness is too small, it is difficult for the particles to have a truly spherical shape, and a sphericity of 0.7 or greater may not be satisfied. When the sphericity is less than 0.7, the effect of the present disclosure cannot be exhibited. In particular, the tactile sensation is extremely deteriorated. Meanwhile, in the case of the particles with too large a degree of surface smoothness, the particles have too few or no pore portions. The particles are hardly deformed by an external force applied thereto, and the tactile sensation (particularly softness) is poor, as a result of which sufficient oil absorption may not be given.

The degree of surface smoothness can be determined as follows: a scanning electron micrograph of the particles is obtained, recesses and protrusions of the particle surfaces are observed, and the degree of surface smoothness is determined based on the area of recessed portions on the surfaces.

Since the total degree of acetyl substitution of cellulose acetate in the cellulose acetate particles of the present disclosure is 0.7 or greater and 3.0 or less, the total degree of acetyl substitution is preferably 0.7 or greater and less than 2.9, more preferably 1.0 or greater and less than 2.9, still more preferably 1.4 or greater and less than 2.9, particularly preferably 1.8 or greater and less than 2.9, and most preferably 1.6 or greater and less than 2.9.

When the total degree of acetyl substitution of the cellulose acetate is less than 0.7, the water solubility increases and cellulose acetate tends to elute in removing the first thermoplastic polymer and the second thermoplastic polymer from the mixture, in the method for producing cellulose acetate particles to be described later. This may reduce the sphericity of the resulting particles and thus may lead to poor tactile sensation.

The total degree of acetyl substitution of the cellulose acetate can be measured by the following method. First, the total degree of acetyl substitution is the sum of each degree of substitution at position 2-, 3-, and 6- of the glucose ring of the cellulose acetate, and each degree of acetyl substitution at position 2-, 3-, and 6- of the glucose ring of the cellulose acetate in the particles can be measured by NMR according to the method of Tezuka (Tezuka, Carbonydr. Res. 273, 83 (1995)). That is, the free hydroxyl group of a cellulose acetate sample is propionylated with propionic anhydride in pyridine. The resulting sample is dissolved in deuterated chloroform, and the 13C-NMR spectrum is measured. The carbon signals of the acetyl group appear in the region from 169 ppm to 171 ppm in the order of 2-, 3-, and 6-positions from the high magnetic field; and the carbonyl carbon signals of the propionyl group appear in the region from 172 ppm to 174 ppm in the same order. Each degree of acetyl substitution at the 2-, 3-, and 6-positions of the glucose ring in the original cellulose acetate can be determined from the presence ratio of the acetyl group and the propionyl group at the respective corresponding positions. The degree of acetyl substitution can be analyzed by ¹H-NMR in addition to ¹³C-NMR.

Furthermore, the total degree of acetyl substitution is determined by converting the acetylation degree determined according to the method for measuring the acetylation degree in ASTM: D-817-91 (Testing methods for cellulose acetate, etc.). This is the most common procedure to determine the degree of substitution of cellulose acetate.

DS=162.14×AV×0.01/(60.052−42.037×AV×0.01)

In the above equation, DS is the total degree of acetyl substitution, and AV is the acetylation degree (%). Note that the value of the degree of substitution calculated by the conversion usually has a slight discrepancy from the value measured by NMR described above. When the converted value and the value measured by NMR are different, the value measured by NMR is adopted. In addition, if the value varies among the specific methods of NMR measurement, the value measured by NMR according to the method of Tezuka described above is adopted.

The method for measuring the acetylation degree according to ASTM: D-817-91 (Testing methods for cellulose acetate, etc.) is outlined as follows. First, 1.9 g of dried cellulose acetate is accurately weighed and dissolved in 150 mL of a mixed solution of acetone and dimethyl sulfoxide (a volume ratio of 4:1), then 30 mL of a 1 N sodium hydroxide solution is added, and the cellulose acetate is saponified at 25° C. for 2 hours. Phenolphthalein is added as an indicator, and the excess sodium hydroxide is titrated with 1N-sulfuric acid (concentration factor: F). In addition, a blank test is performed in the same manner as described above, and the acetylation degree is calculated according to the following equation.

Average acetylation degree (%)={6.5×(B−A)×F}/W

where A represents the titration volume (mL) of the 1 N sulfuric acid for the sample, B represents the titration volume (mL) of the 1 N sulfuric acid for the blank test, F represents the concentration factor of the 1 N sulfuric acid, and W represents the weight of the sample.

The cellulose acetate particles of the present disclosure may have a bulk specific gravity of 0.1 or greater and 0.9 or less, 0.2 or greater and 0.9 or less, or 0.2 or greater and 0.7 or less. For example, for a cosmetic containing the particles, the higher the bulk specific gravity of the particles, the better the flowability of the cosmetic composition is. The bulk specific gravity can be measured by a method in accordance with JIS K 1201-1.

The oil absorption of the cellulose acetate particles of the present disclosure using linseed oil is preferably 60 ml or greater, more preferably 70 ml or greater, and still more preferably 80 ml or greater per 100 g of the cellulose acetate particles. The oil absorption may be 200 ml or less, and is preferably 100 ml or less, more preferably 90 ml or less. When the oil absorption is 60 ml or greater per 100 g of the cellulose acetate particles, the cellulose acetate particles are particularly excellent in tactile sensation, for example, a cosmetic composition containing the cellulose acetate particles provides improved skin feel. When the oil absorption is more than 200 ml per 100 g of the cellulose acetate particles, the cosmetic composition absorbs the oil content of skin more than necessary, which may cause dryness of the skin. From the viewpoint of preventing excessive dryness of the skin and preparing a cosmetic composition giving good skin feel, the oil absorption may be from 60 ml to 200 ml, from 60 ml to 100 ml, from 60 ml to 90 ml, from 70 ml to 200 ml, from 70 ml to 100 ml, from 70 ml to 90 ml, from 80 ml to 200 ml, from 80 ml to 100 ml, or from 80 ml to 90 ml per 100 g of the cellulose acetate particles.

The oil absorption using linseed oil can be determined by Test methods for pigments, specified in JIS K 5101-13-1: 2004 (ISO 787-5: 1980)—Part 13: Oil absorption—Section 1: Refined linseed oil method.

The cellulose acetate particles of the present disclosure may contain or need not contain a plasticizer. In the present disclosure, the plasticizer refers to a compound capable of increasing the plasticity of the cellulose acetate. The plasticizer is not particularly limited, and examples include adipate-based plasticizers containing an adipate ester, such as dimethyl adipate, dibutyl adipate, diisostearyl adipate, diisodecyl adipate, diisononyl adipate, diisobutyl adipate, diisopropyl adipate, diethylhexyl, adipate dioctyl adipate, dioctyldodecyl adipate, dicapryl adipate, and dihexyldecyl adipate; citrate-based plasticizers containing a citrate ester, such as acetyl triethyl citrate, acetyl tributyl citrate, isodecyl citrate, isopropyl citrate, triethyl citrate, triethylhexyl citrate, and tributyl citrate; glutarate-based plasticizers containing a glutarate ester, such as diisobutyl glutarate, dioctyl glutarate, and dimethyl glutarate; succinate-based plasticizers containing a succinate ester, such as diisobutyl succinate, diethyl succinate, diethylhexyl succinate, and dioctyl succinate; sebacate-based plasticizers containing a sebacate ester, such as diisoamyl sebacate, diisooctyl sebacate, diisopropyl sebacate, diethyl sebacate, diethylhexyl sebacate, and dioctyl sebacate; glycerin ester-based plasticizers containing a glycerin alkyl ester, such as triacetin, diacetin, and monoacetin; neopentyl glycol; phthalate-based plasticizers containing a phthalate ester, such as ethyl phthalate, methyl phthalate, diaryl phthalate, diethyl phthalate, diethylhexyl phthalate, dioctyl phthalate, dibutyl phthalate, and dimethyl phthalate; and phosphate-based plasticizers containing a phosphate ester, such as trioleil phosphate, tristearyl phosphate, and tricetyl phosphate. In addition, examples thereof also include di-2-methoxyethyl phthalate, dibutyl tartrate, ethyl 0-benzoylbenzoate, ethyl phthalyl ethyl glycolate (EPEG), methyl phthalyl ethyl glycolate (MPEG), N-ethyl toluene sulfonamide, 0-cresyl p-toluenesulfonate, triethyl phosphate (TEP), triphenyl phosphate (TPP), and tripropionin. These plasticizers may be used alone, or two or more of the plasticizers may be used in combination.

Among the plasticizers, the plasticizer is preferably at least one selected from the group consisting of citrate-based plasticizers containing a citrate ester, such as triethyl citrate, acetyl triethyl citrate, and acetyl tributyl citrate; glycerin ester-based plasticizers containing a glycerin alkyl ester, such as triacetin, diacetin, and monoacetin; and phthalate-based plasticizers, such as ethyl phthalate and methyl phthalate; more preferably at least one selected from the group consisting of triethyl citrate, acetyl triethyl citrate, acetyl tributyl citrate, triacetin, diacetin, diisononyl adipate, and diethyl phthalate; still more preferably at least one selected from the group consisting of acetyl triethyl citrate, triacetin, diacetin, and diethyl phthalate; and particularly preferably at least one selected from the group consisting of acetyl triethyl citrate and triacetin. Note that a phthalate-based plasticizer must be used with care because of concerns about similarity to environmental hormones.

When the cellulose acetate particles contain a plasticizer, the content of the plasticizer included in the cellulose acetate particles is not particularly limited. For example, the content of the plasticizer may be more than 0 parts by weight and 67 parts by weight or less, 2 parts by weight or greater and 67 parts by weight or less, 11 parts by weight or greater and 43 parts by weight or less, or 18 parts by weight or greater and 25 parts by weight or less relative to 100 parts by weight of the cellulose acetate.

The content rate of the plasticizer in the cellulose acetate particles is determined by ¹H-NMR measurement.

The cellulose acetate particles of the present disclosure have excellent biodegradability. The biodegradation rate is preferably 40 wt. % or greater, more preferably 50 wt. % or greater, and still more preferably 60 wt. % or greater within 30 days.

The biodegradation rate can be measured by a method using activated sludge in accordance with JIS K6950.

The cellulose acetate particles of the present disclosure can be produced by a production method described later.

The cellulose acetate particles of the present disclosure are excellent in biodegradability, tactile sensation, and oil absorbability, and thus can be suitably used, for example, in cosmetic compositions. In a cosmetic composition containing the cellulose acetate particles of the present disclosure, the oil absorption of the cellulose acetate particles is high, and thus the cellulose acetate particles can absorb sebum and prevent makeup from coming off. Further, the cellulose acetate particles of the present disclosure have a high sphericity, are likely to be deformed by an external force applied thereto, and are excellent in softness. Accordingly, the cellulose acetate particles can improve the tactile sensation of the cosmetic composition. Additionally, since the cellulose acetate particles of the present disclosure have many pores, it is easy to cause the pores to carry a functional agent or the like, and the cellulose acetate particles can be suitably used as functional particles.

Examples of the cosmetic composition include foundation, such as liquid foundation and powder foundation; concealers; sunscreens; makeup bases; lipsticks and lipstick bases; Oshiroi face powders, such as body powders, solid face powders, and face powders; solid powder eye shadows; wrinkle masking creams; and skin and hair external preparations mainly for cosmetic purposes, such as skin care lotions; and the dosage form is not limited. The dosage form may be any of a liquid preparation, such as an aqueous solution, a milky lotion, and a suspension; a semi-solid preparation, such as a gel and a cream; or a solid preparation, such as a powder, a granule, and a solid. In addition, the dosage form may be an emulsion preparation, such as a cream and a milky lotion; an oil gel preparation, such as a lipstick; a powder preparation, such as a foundation; an aerosol preparation, such as a hair styling agent; or the like.

### Method for Producing Cellulose Acetate Particles

A method for producing cellulose acetate particles of the present disclosure is as follows. A method for producing cellulose acetate particles includes: mixing cellulose acetate having a total degree of acetyl substitution of 0.7 or greater and 3.0 or less, a plasticizer, a first thermoplastic polymer, and a second thermoplastic polymer to form a mixture of cellulose acetate containing the plasticizer, the first thermoplastic polymer, and the second thermoplastic polymer; melt-kneading the mixture at 200° C. or higher and 280° C. or lower; and removing the first thermoplastic polymer and the second thermoplastic polymer from the melt-kneaded mixture, in which when SPa represents an SP value of the cellulose acetate, SPb represents an SP value of the first thermoplastic polymer, and SPc represents an SP value of the second thermoplastic polymer, SPa, SPb, and SPc satisfy the following relationship:

0.1≤|SPc−SPa|/|SPb−SPa|≤0.9.

### Formation of Mixture of Cellulose Acetate

Cellulose acetate having a total degree of acetyl substitution of 0.7 or greater and 3.0 or less, a plasticizer, a first thermoplastic polymer, and a second thermoplastic polymer are mixed in the formation of a mixture of cellulose acetate containing the plasticizer, the first thermoplastic polymer, and the second thermoplastic polymer.

The cellulose acetate having a total degree of acetyl substitution of 0.7 or greater and 3.0 or less can be produced by a well-known method for producing cellulose acetate. Examples of such a production method include what is called an acetic acid method in which acetic anhydride is used as an acetylating agent, acetic acid as a diluent, and sulfuric acid as a catalyst. The basic processes of the acetic acid method include: (1) pretreatment including grinding/disintegrating a pulp raw material (soluble pulp) having a relatively high α-cellulose content and then spraying acetic acid and mixing them; (2) acetylation including reacting the pretreated pulp from (1) with a mixed acid containing acetic anhydride, acetic acid, and an acetylation catalyst (e.g., sulfuric acid); (3) aging including hydrolyzing cellulose acetate to form cellulose acetate having a desired acetylation degree; and (4) post-treatment including precipitating the cellulose acetate to separate it from the reaction solution after completion of the hydrolysis reaction, then purifying, stabilizing, and drying the cellulose acetate.

Since the total degree of acetyl substitution of the cellulose acetate is 0.7 or greater and 3.0 or less, the total degree of acetyl substitution is preferably 0.7 or greater and less than 2.9, more preferably 1.0 or greater and less than 2.9, still more preferably 1.4 or greater and less than 2.9, particularly preferably 1.8 or greater and less than 2.9, and most preferably 1.6 or greater and less than 2.9. The total degree of acetyl substitution can be adjusted by adjusting the conditions of aging (conditions, such as time and temperature).

Any plasticizer having a plasticizing effect in melt-extruding cellulose acetate can be used without particular limitation. Specifically, the plasticizers exemplified as a plasticizer contained in the cellulose acetate particles may be used alone or two or more of the plasticizers may be used in combination.

Among the plasticizers exemplified above, the plasticizer is preferably at least one selected from the group consisting of citrate-based plasticizers containing a citrate ester, such as triethyl citrate, acetyl triethyl citrate, and acetyl tributyl citrate; glycerin ester-based plasticizers containing a glycerin alkyl ester, such as triacetin, diacetin, and monoacetin; adipate-based plasticizers, such as diisononyl adipate; and phthalate-based plasticizers, such as ethyl phthalate and methyl phthalate; more preferably at least one selected from the group consisting of triethyl citrate, acetyl triethyl citrate, acetyl tributyl citrate, triacetin, diacetin, diisononyl adipate, and diethyl phthalate; still more preferably at least one selected from the group consisting of acetyl triethyl citrate, triacetin, diacetin, and diethyl phthalate; and particularly preferably at least one selected from the group consisting of acetyl triethyl citrate and triacetin. Note that a phthalate-based plasticizer must be used with care because of concerns about similarity to environmental hormones.

The blending amount of the plasticizer may be more than 0 parts by weight and 67 parts by weight or less, 2 parts by weight or greater and 67 parts by weight or less, 11 parts by weight or greater and 43 parts by weight or less, or 18 parts by weight or greater and 25 parts by weight or less relative to 100 parts by weight of the cellulose acetate. Too small blended amount of the plasticizer would tend to reduce the sphericity of the resulting cellulose acetate particles, and too large blended amount of the plasticizer would fail to maintain the shape of the particles, tending to reduce the sphericity.

The first thermoplastic polymer and the second thermoplastic polymer can be used without any particular limitation as long as they satisfy the relationship below.

When SPa represents an SP value of the cellulose acetate, SPb represents an SP value of the first thermoplastic polymer, and SPc represents an SP value of the second thermoplastic polymer, SPa, SPb, and SPc satisfy the following relationship:

0.1≤|SPc−SPa|/|SPb−SPa|≤0.9.

The thermoplastic polymer in the present specification is not particularly limited as long as it is a polymer having a wide range of thermoplasticity. Both the first thermoplastic polymer and the second thermoplastic polymer preferably have water solubility, in other words, are preferably water-soluble polymers. Here, the water-soluble polymer means that an insoluble content is less than 50 wt. % when 1 g of the polymer is dissolved in 100 g of water at 25° C. Further, the “polymer” is defined as a compound having a structure formed by repeatedly bonding one or two or more of constituent units. In the present specification of the present application, a compound having a weight-average molecular weight of 10000 or greater is referred to as a “polymer”.

Examples of the first thermoplastic polymer or the second thermoplastic polymer include polyvinyl alcohol, polyethylene glycol, sodium polyacrylate, polyvinylpyrrolidone, polypropylene oxide, polyglycerol, polyethylene oxide, polyvinyl acetate, modified starch, thermoplastic starch, methylcellulose, ethylcellulose, hydroxyethyl cellulose, and hydroxypropylcellulose. Further, the thermoplastic starch can be prepared by a well-known method. For example, reference can be made to JP H06-006307 B, WO 92/04408, etc., and more specifically, for example, a thermoplastic starch prepared by mixing approximately 20% of glycerin as a plasticizer to tapioca starch and kneading them with a twin-screw extruder can be used.

The first thermoplastic polymer preferably contains at least one selected from the group consisting of polyvinyl alcohol, sodium polyacrylate, polyvinylpyrrolidone, and thermoplastic starch, and more preferably contains at least one selected from the group consisting of polyvinyl alcohol and thermoplastic starch. Further, the weight-average molecular weight of polyvinyl alcohol is preferably 500 or greater and 50000 or less.

The second thermoplastic polymer may be a thermoplastic polymer that satisfies 0.1≤|SPc−SPa|/|SPb−SPa|≤0.9. Preferred is a thermoplastic polymer that satisfies 0.2<|SPc−SPa|/|SPb−SPa|<0.8. When this value is |SPc−SPa|/|SPb−SPa|<0.1 or |SPc−SPa|/| SPb−SPa|>0.9, the size of pores formed in the resulting cellulose acetate particles is small, the number of pores is also small, the relative specific surface area (RSSA) is reduced, and the tactile sensation is poor. Thus, this is not preferred.

The second thermoplastic polymer is preferably polyethylene glycol.

When polyvinyl alcohol, thermoplastic starch, or modified starch is used as the first thermoplastic polymer, it is particularly preferable to use polyethylene glycol as the second thermoplastic polymer. This is because polyvinyl alcohol, thermoplastic starch, modified starch, and polyethylene glycol are all water-soluble and thermoplastic. Further, the weight-average molecular weight of polyethylene glycol is preferably 500 or greater and 50000 or less.

Here, the weight-average molecular weight (Mw) is a value obtained by multiplying individual molecules by their molecular weights and taking the weighted average, and is determined by gel permeation chromatography (GPC).

The blending amount of the first thermoplastic polymer is preferably 110 parts by weight or greater and 15000 parts by weight or less, more preferably 180 parts by weight or greater and 1200 parts by weight or less, still more preferably 200 parts by weight or greater and 800 parts by weight or less relative to 100 parts by weight of the cellulose acetate. When the blending amount is less than 110 parts by weight, cellulose acetate particles having poor sphericity, i.e., having a non-spherical modified shape, may be produced. When the blending amount is more than 15000 parts by weight, the particle size of the resulting cellulose acetate particles may be too small.

The blending amount of the second thermoplastic polymer is preferably 1 part by weight or greater and 1500 parts by weight or less, more preferably 2 parts by weight or greater and 150 parts by weight or less, still more preferably 3 parts by weight or greater and 100 parts by weight or less relative to 100 parts by weight of the cellulose acetate. When the blending amount is less than 1 part by weight, sufficient pores are not formed in the resulting cellulose acetate particles, and the oil absorption may become insufficient.

Mixing of the cellulose acetate and the plasticizer, or mixing of the cellulose acetate, the plasticizer, the first thermoplastic polymer, and the second thermoplastic polymer can be performed in a dry or wet manner using a mixer such as a Henschel mixer. In using a mixer, such as a Henschel mixer, the temperature in the mixer may be a temperature at which the cellulose acetate does not melt, for example, in a range of not lower than 20° C. and lower than 200° C.

Alternatively, mixing of the cellulose acetate and the plasticizer, or mixing of the cellulose acetate, the plasticizer, the first thermoplastic polymer, and the second thermoplastic polymer may be performed by melt-kneading. Furthermore, the melt-kneading may be performed in combination with mixing using a mixer, such as a Henschel mixer, and in this case, the melt-kneading is preferably performed after mixing in temperature conditions in a range of not lower than 20° C. and lower than 200° C. using a mixer, such as a Henschel mixer. The plasticizer and the cellulose acetate, or the plasticizer, the first thermoplastic polymer, the second thermoplastic polymer, and the cellulose acetate become more uniform and mixed well in a short period of time, thus increasing the sphericity of the cellulose acetate particles that are finally prepared and improving the tactile sensation and touch feeling of the particles.

The melt-kneading is preferably performed by heating and mixing with an extruder. The kneading temperature (cylinder temperature) of the extruder may be in a range of 200° C. to 230° C. The melt-kneading performed even at temperatures in this range can plasticize the cellulose acetate and provide a uniform kneaded product. The melt-kneading performed at too low temperatures may reduce the sphericity of the resulting particles, thus reducing the tactile sensation and the touch feeling. The melt-kneading performed at too high temperatures may cause deterioration or coloration of the kneaded product due to heat and may reduce the viscosity of the melted material, thus failing to sufficiently knead the resin in a twin-screw extruder.

The melting point of the cellulose acetate depends on the degree of substitution but is approximately from 230° C. to 280° C. and is close to the decomposition temperature of the cellulose acetate. Thus, melt-kneading is typically difficult in this temperature range, but because the cellulose acetate (flakes) impregnated with the plasticizer can reduce the plasticizing temperature. The kneading temperature (cylinder temperature) may be, for example, 200° C. in using a twin-screw extruder. The kneaded product may be extruded in a strand shape and formed into a pellet form by hot cutting or the like. The die temperature in this case may be approximately 220° C.

### Melt-kneading of Mixture of Cellulose Acetate

In the present step, the mixture is melt-kneaded at 200° C. or higher and 280° C. or lower.

The mixture can be kneaded by an extruder such as a twin-screw extruder. Further, the temperature of the kneading refers to the cylinder temperature.

The mixture of cellulose acetate may be extruded into a string shape from a die attached to the tip of an extruder, such as a twin-screw extruder, and then cut into pellets. At this time, the die temperature may be not lower than 220° C. and not higher than 300° C.

### Removal of First Thermoplastic Polymer and Second Thermoplastic Polymer

The removal of the first thermoplastic polymer and the second thermoplastic polymer from the melt-kneaded mixture will be described.

The method of removing the first thermoplastic polymer and the second thermoplastic polymer is not particularly limited as long as the first thermoplastic polymer and the second thermoplastic polymer can be removed from the melt-kneaded mixture by dissolution or the like. Examples thereof include a method of removing the first thermoplastic polymer and the second thermoplastic polymer from the mixture by dissolving the thermoplastic polymers in water; an alcohol such as methanol, ethanol, or isopropanol; or a solvent such as a mixed solution thereof. Specifically, examples thereof include a method of removing the first thermoplastic polymer and the second thermoplastic polymer from the mixture, such as by mixing the mixture and the solvent and filtering the mixture to take out the filtrate.

In removing the first thermoplastic polymer and the second thermoplastic polymer from the mixture, the plasticizer may be removed, or need not be removed from the mixture together with the first thermoplastic polymer and the second thermoplastic polymer. Thus, the resulting cellulose acetate particles may or may not contain a plasticizer.

Regarding the mixing ratio of the mixture and the solvent, the mixture is preferably 0.01 wt. % or greater and 20 wt. % or less, more preferably 2 wt. % or greater and 15 wt. % or less, and still more preferably 4 wt. % or greater and 13 wt. % or less relative to the total weight of the mixture and the solvent. When the mixing ratio of the mixture is more than 20 wt. %, dissolution of the first and second thermoplastic polymers becomes insufficient and the first and second thermoplastic polymers cannot be removed by washing, or it may be difficult to separate the cellulose acetate particles not dissolved in the solvent from the first and second thermoplastic polymers dissolved in the solvent by an operation such as filtration or centrifugal separation.

The mixing temperature of the mixture and the solvent is preferably 0° C. or higher and 200° C. or lower, more preferably 20° C. or higher and 110° C. or lower, and still more preferably 40° C. or higher and 80° C. or lower. At temperatures lower than 0° C., the first thermoplastic polymer and the second thermoplastic polymer may not be sufficiently dissolved and it may be difficult to remove them by washing, whereas, at temperatures higher than 200° C., deformation, aggregation, or the like of the particles may occur, and it may be difficult to take out the particles while maintaining the desired shape of the particles.

The mixing time of the mixture and the solvent is not particularly limited, and may be appropriately adjusted, but may be, for example, 0.5 hours or longer, 1 hour or longer, 3 hours or longer, 5 hours or longer, or may be 6 hours or shorter.

In addition, the method of mixing is not limited as long as the method can dissolve the first thermoplastic polymer and the second thermoplastic polymer, but, for example, use of a stirring device, such as an ultrasonic homogenizer or a Three-One Motor, can efficiently remove the first thermoplastic polymer and the second thermoplastic polymer from the mixture even at room temperature.

For example, when a Three-One Motor is used as the stirring device, the rotation speed during mixing the mixture and the solvent may be, for example, 5 rpm or greater and 3000 rpm or less. This enables the first thermoplastic polymer and the second thermoplastic polymer to be efficiently removed from the mixture. In addition, this also efficiently removes the plasticizer from the mixture.

### Emulsifiable Preparation

The emulsifiable preparation of the present disclosure (hereinafter, sometimes referred to as “emulsifiable preparation”) is a Pickering emulsion. The emulsifiable preparation is produced by an emulsification method using microparticles as solid particles. The emulsifiable preparation includes an aqueous component, an oily component, and microparticles. The microparticles adsorb to the emulsifying interface between the aqueous component and the oily component. The oily component in a state of being covered with the microparticles is dispersed in the aqueous component. This makes it possible to stably maintain a state in which the oily component is uniformly dispersed in the aqueous component. Therefore, an emulsifiable preparation being in excellent storage stability is produced. Hereinafter, the microparticles, the aqueous component, and the oily component will be described in detail.

### Microparticles

In the emulsifiable preparation of the present disclosure, microparticles of a polymer compound contain cellulose acetate as the polymer compound. Since cellulose acetate is a polymer compound, cellulose acetate is hardly absorbed from the skin and intestinal wall, and is safe for the human body. From a long-time perspective, cellulose acetate, i.e., a biodegradable polymer compound, is decomposed even when it is released in nature, and thus the influence on the environment can be suppressed.

The content of the polymer compound contained in the microparticles is preferably 60 wt. % or greater, more preferably 80 wt. % or greater, and still more preferably 95 wt. % or greater relative to the total weight of the microparticles. When the content of the polymer compound contained in the microparticles is 60 wt. % or greater relative to the total weight of the microparticles, microparticles are more likely to have a well-ordered shape.

In addition, the microparticles are not limited to cellulose acetate, and may contain one or more other polymer compounds. The microparticles may be, for example, particles of cellulose acetate coated with another polymer compound, may be particles of another polymer compound coated with cellulose acetate, or may be particles formed of a mixture of cellulose acetate and another polymer compound. When the microparticles contain another polymer compound, the microparticles can have properties that are not possessed by cellulose acetate. Thus, for example, the taste, color, and feeling to the touch of the emulsifiable preparation can vary depending on the intended use.

The content of cellulose acetate contained in the polymer compound of the microparticles is preferably 40 wt. % or greater, more preferably 60 wt. % or greater, and still more preferably 80 wt. % or greater relative to the total weight of the polymer compound.

Another polymer compound contained in the microparticles is preferably a biodegradable polymer compound. Examples of the biodegradable polymer compound include polylactic acid, polyglycolic acid, polyaspartic acid, polyvinyl alcohol, polyhydroxyalkanoate, modified polyethylene terephthalate, starch (glucose polymer), cellulose derivatives other than cellulose acetate, polybutylene succinate-based compounds, polycaprolactone, and gelatin. When the emulsifiable preparation is used for foods or beverages, another polymer compound may be, for example, an edible polysaccharide. Examples of the polysaccharide include pullulan, gellan gum, xanthan gum, tamarind seed gum, locust bean gum, pectin, carrageenan, guar gum, gum arabic, dextran, dextrin, sodium chondroitin sulfate, sodium hyaluronate, and sodium alginate. When the emulsifiable preparation is used for cosmetics, examples of another polymer compound include polyvinylpyrrolidone, carboxyvinyl polymer, sodium polyacrylate, methacrylic acid copolymer, and polyethylene glycol.

The microparticles have an average particle size of 2 to 10 μm. The upper limit of the average particle size of the microparticles is preferably 8 μm, and more preferably 7 μm. The lower limit of the average particle size of the microparticles is preferably 4 μm, and more preferably 5 μm. The microparticles have an average particle size of 2 to 10 μm, and thus the microparticles are not caught in wrinkles on the skin when the emulsifiable preparation is applied to the skin, and the touch feeling to the skin after application is improved. In addition, when the microparticles have an average particle size of 2 to 10 μm, the storage stability of the emulsifiable preparation is improved. Furthermore, when the average particle size of the microparticles is 5 μm or greater, the microparticles can impart a pleasant touch feeling to the emulsifiable preparation due to a ball-bearing effect.

The average particle size can be measured using dynamic light scattering. The specific measurement procedure is as follows. First, the microparticles are suspended at a concentration of 100 ppm in pure water using an ultrasonic vibrating apparatus to prepare a sample. Then, the average particle size can be derived from the particle size volume distribution measured by laser diffraction (“Laser Diffraction/Scattering Particle Size Distribution Measuring Apparatus LA-960” available from Horiba Ltd., ultrasonic treatment for 15 minutes, and a refractive index (1.500, medium (water; 1.333)). The average particle size (such as in nm and μm) herein refers to the value of the particle size corresponding to 50% of the integrated scattering intensity in this particle size distribution.

Microparticles containing cellulose acetate as a polymer compound (hereinafter, sometimes referred to as “cellulose acetate microparticles”) can be prepared by the following method (see WO 2019/156116 A1). A method for producing cellulose acetate microparticles includes: preparing cellulose acetate impregnated with a plasticizer; preparing a dispersion containing, as a dispersoid, the cellulose acetate impregnated with the plasticizer; and removing a water-soluble polymer from the prepared dispersion. Note that the cellulose acetate microparticles are not limited to being prepared by the following method, and may be prepared by another known method.

The coefficient of variation of the particle size of the cellulose acetate microparticles of the present disclosure is preferably 0% or greater and 60% or less, and more preferably 2% or greater and 50% or less. The coefficient of variation (%) of the particle size can be calculated by an equation: (standard deviation of particle size)/(average particle size)×100. When the coefficient of variation of the particle size of the cellulose acetate microparticles is 0% or greater and 60% or less, the particle size of the cellulose acetate microparticles is within a certain range. Therefore, when the cellulose acetate microparticles are used in the aqueous cosmetic material, the feeling to the touch of the aqueous cosmetic material is improved.

The sphericity of the cellulose acetate microparticles of the present disclosure is 0.7 or greater and 1.0 or less, preferably 0.8 or greater and 1.0 or less, and more preferably 0.9 or greater and 1.0 or less. When the sphericity of the cellulose acetate microparticles is 0.7 or greater and 1.0 or less, the feeling to the touch of the microparticles is excellent. Therefore, even when the cellulose acetate microparticles are used in the aqueous cosmetic material, the feeling to the touch of the aqueous cosmetic material and the soft focus effect are improved.

The sphericity can be measured by the following method. 30 particles are randomly selected from an image of cellulose acetate microparticles observed with a scanning electron microscope (SEM), the major axis length and the minor axis length of the selected particles are measured, and the ratio (minor axis length)/(major axis length) of each particle is determined. Then, the average value of the ratios (minor axis length)/(major axis length) is defined as the sphericity. The closer to 1 the sphericity is, the closer to the true sphere the particles can be determined to be.

Preparation of Cellulose Acetate Impregnated with Plasticizer

The cellulose acetate impregnated with the plasticizer is prepared by mixing the cellulose acetate and the plasticizer. The total degree of acetyl substitution of cellulose acetate is preferably 0.7 or greater and 2.9 or less, more preferably 1.4 or greater and less than 2.6, and still more preferably 2.0 or greater and less than 2.6. When the total degree of acetyl substitution is 0.7 or greater and 2.9 or less, cellulose acetate is less soluble in water, and thus the sphericity of the particles to be produced increases. The particles also have sufficiently high biodegradability. The cellulose acetate having a total degree of acetyl substitution of 0.7 or greater and 2.9 or less is produced by a known method for producing cellulose acetate. Examples of such a known method for producing cellulose acetate include a so-called acetic acid method in which acetic anhydride is used as an acetylating agent, acetic acid is used as a diluent, and sulfuric acid is used as a catalyst.

The plasticizer can be used without any particular limitation as long as it has a plastic effect in the process of melting and extruding cellulose acetate. For example, the plasticizer is preferably at least one selected from the group consisting of citrate-based plasticizers containing a citrate ester, such as triethyl citrate, acetyl triethyl citrate, and acetyl tributyl citrate; glycerin ester-based plasticizers containing a glycerin alkyl ester, such as triacetin, diacetin, and monoacetin; adipate-based plasticizers, such as diisononyl adipate; and phthalate-based plasticizers, such as ethyl phthalate and methyl phthalate; more preferably at least one selected from the group consisting of triethyl citrate, acetyl triethyl citrate, acetyl tributyl citrate, triacetin, and diisononyl adipate; and even more preferably at least one selected from the group consisting of acetyl triethyl citrate, triacetin, diacetin, and diethyl phthalate. The single plasticizer can be used alone, or the two or more plasticizers can be used in combination.

The blending amount of the plasticizer is preferably more than 0 part by weight and 40 parts by weight or less, more preferably 2 parts by weight or greater and 40 parts by weight or less, still more preferably 10 parts by weight or greater and 30 parts by weight or less, and most preferably 15 parts by weight or greater and 20 parts by weight or less relative to 100 parts by weight of the total amount of the cellulose acetate and the plasticizer. When the plasticizer is blended in an amount of more than 0 part by weight and 40 parts by weight or less relative to 100 parts by weight of the total amount of the cellulose acetate and the plasticizer, the sphericity of the cellulose acetate microparticles to be produced increases.

The cellulose acetate and the plasticizer can be dry-mixed or wet-mixed using an existing mixer, such as a Henschel mixer. When a mixer is used, the temperature inside the mixer is preferably in a range of 20° C. or higher and less than 200° C. When the temperature inside the mixer is 20° C. or higher and less than 200° C., the melting of the cellulose acetate can be prevented. In addition, the cellulose acetate and the plasticizer may be mixed by melt-kneading. The melt-kneading may be performed in combination with mixing using a mixer, and in this case, the melt-kneading is preferably performed after mixing in temperature conditions in a range of 20° C. or higher and lower than 200° C. using a mixer. This allows the plasticizer and cellulose acetate to be mixed more uniformly in a short period of time. Therefore, the sphericity of the cellulose acetate microparticles to be finally prepared increases, and the tactile sense and feeling to the touch of microparticles are improved.

The melt-kneading is performed by heating and mixing with an extruder. Preferably, the kneading temperature (cylinder temperature) of the extruder is in a range of 200° C. to 230° C. The melting point of cellulose acetate is approximately in a range of 230° C. to 280° C., although it varies depending on the degree of substitution. Here, the plasticization temperature of the cellulose acetate impregnated with the plasticizer is reduced. Thus, even when the kneading temperature of the extruder is in a range of 200° C. to 230° C., it is possible to obtain a uniform kneaded product in which the cellulose acetate is plasticized. Note that the kneading temperature may be, for example, 200° C. in using a twin-screw extruder. The kneaded product may be extruded in a strand shape and formed into a pellet form by hot cutting or the like. The die temperature in this case may be, for example, approximately 220° C.

### Preparation of Dispersion

In preparing the dispersion, the cellulose acetate impregnated with the plasticizer and a water-soluble polymer are first kneaded. The blending amount of the water-soluble polymer is preferably 55 parts by weight or greater and 99 parts by weight or less, more preferably 60 parts by weight or greater and 90 parts by weight or less, and still more preferably 65 parts by weight or greater and 85 parts by weight or less relative to 100 parts by weight of the total amount of the cellulose acetate impregnated with the plasticizer and the water-soluble polymer.

The water-soluble polymer used in preparing a dispersion refers to a polymer having an insoluble content of less than 50 wt. % when 1 g of the polymer is dissolved in 100 g of water at 25° C. Examples of the water-soluble polymer may include polyvinyl alcohol, polyethylene glycol, sodium polyacrylate, polyvinylpyrrolidone, polypropylene oxide, polyglycerin, polyethylene oxide, vinyl acetate, modified starch, thermoplastic starch, methyl cellulose, ethyl cellulose, hydroxyethyl cellulose, and hydroxypropyl cellulose. Among them, polyvinyl alcohol, polyethylene glycol, and thermoplastic starch are preferred, and polyvinyl alcohol and thermoplastic starch are particularly preferred. Further, the thermoplastic starch can be prepared by a well-known method.

The kneading of the cellulose acetate impregnated with the plasticizer and the water-soluble polymer can be performed, for example, with an extruder such as a twin-screw extruder. The cellulose acetate impregnated with the plasticizer and the water-soluble polymer are kneaded at 200° C. or higher and 280° C. or lower. The temperature of the kneading refers to the cylinder temperature of the extruder. The kneaded product is extruded into a string shape from the die attached to the tip of the extruder. At this time, the die temperature may be not lower than 220° C. and not higher than 300° C. The kneaded product extruded into a string shape is cut and formed into a pellet form to prepare a dispersion. The resulting dispersion is a dispersion containing: the water-soluble polymer as a dispersion medium; and the cellulose acetate impregnated with the plasticizer as a dispersoid.

### Removal of Water-Soluble Polymer

The removal of the water-soluble polymer from the dispersion is described. The method of removing the water-soluble polymer is not particularly limited as long as the water-soluble polymer dissolves and can be removed from the particles. Examples of the method of removing the water-soluble polymer include a method of dissolving and removing the water-soluble polymer of the dispersion using a solvent for removal of the water-soluble polymer, such as water, an alcohol, such as methanol, ethanol, or isopropanol, or their mixed solution. Specifically, examples include a method of removing the water-soluble polymer from the dispersion, such as by mixing the dispersion and the solvent for removal of the water-soluble polymer and filtering the mixture to take out the filtrate.

The mixing ratio of the dispersion and the solvent for removal of the water-soluble polymer is preferably 0.01 wt. % or greater and 20 wt. % or less, more preferably 2 wt. % or greater and 15 wt. % or less, and still more preferably 4 wt. % or greater and 13 wt. % or less relative to the total weight of the dispersion and the solvent for removal of the water-soluble polymer. When the content of the dispersion is 0.01 wt. % or greater and 20 wt. % or less relative to the total weight of the dispersion and the solvent for removal of the water-soluble polymer, the water-soluble polymer is sufficiently dissolved in the solvent, and the water-soluble polymer can be sufficiently washed and removed. In addition, it is possible to easily separate the cellulose acetate microparticles that are not dissolved in the solvent for removal of the water-soluble polymer and the water-soluble polymer that is dissolved in the solvent for removal of the water-soluble polymer, by an operation such as filtration or centrifugation.

The mixing temperature of the dispersion and the solvent for removal of the water-soluble polymer is preferably 0° C. or higher and 200° C. or lower, more preferably 20° C. or higher and 110° C. or lower, and still more preferably 40° C. or higher and 80° C. or lower. When the mixing temperature of the dispersion and the solvent for removal of the water-soluble polymer is 0° C. or higher and 200° C. or lower, the water-soluble polymer is sufficiently dissolved in the solvent for removal of the water-soluble polymer, and the water-soluble polymer can be sufficiently washed and removed. Further, the particles can be taken out while maintaining a desired shape of the particles without causing deformation, aggregation, or the like of the particles.

The mixing time of the dispersion and the solvent for removal of the water-soluble polymer is not particularly limited and may be appropriately adjusted, but may be, for example, 0.5 hours or more, 1 hour or more, 3 hours or more, or 5 hours or more, and may be 6 hours or less.

In addition, the method of mixing the dispersion and the solvent for removal of the water-soluble polymer is not limited as long as the water-soluble polymer can be dissolved, but, for example, a stirring device, such as an ultrasonic homogenizer or a Three-One Motor, can efficiently remove the water-soluble polymer from the dispersion even at room temperature. For example, when a Three-One Motor is used as the stirring device, the rotation speed during mixing the dispersion and the solvent may be, for example, not less than 5 rpm and not greater than 3000 rpm. This can more efficiently remove the water-soluble polymer from the dispersion. In addition, this also efficiently removes the plasticizer from the dispersion. This results in microparticles containing cellulose acetate (cellulose acetate microparticles).

In removing the water-soluble polymer from the dispersion, the plasticizer may be or need not be removed from the dispersion together with the water-soluble polymer. Thus, the resulting cellulose acetate microparticles may contain or need not contain a plasticizer.

### Aqueous Component

In the emulsifiable preparation of the present disclosure, the aqueous component contains one or more aqueous components selected from the group consisting of water and alcohols. In the present specification, the aqueous component refers to water or a water-soluble component. Since the emulsifiable preparation contains the aqueous component, the emulsifiable preparation of the present disclosure provides a refreshing feeling to the touch.

The alcohol contained in the aqueous component is not particularly limited, but is preferably an alcohol that is highly safe to the human body. As the alcohol, for example, an alcohol that can be used as an oral preparation or an alcohol that is safe when applied to the skin can be adopted. One type of alcohol may be contained, or two or more types thereof may be contained. The type and content of the alcohol are appropriately selected according to the use, the solubility of the additive, or the like.

Examples of the alcohol include lower alcohols having from 1 to 4 carbons, such as ethanol or isopropyl alcohol, or polyhydric alcohols. Examples of the polyhydric alcohols include propylene glycol, dipropylene glycol, glycerin, diglycerin, low-molecular-weight (e.g., a weight-average molecular weight of 1000 or greater and 20000 or less) polyethylene glycol, 1,3-butylene glycol, and 1,2-pentanediol. Polyhydric alcohols have low volatility and are used as lubricants. Thus, when the aqueous component contains a polyhydric alcohol, the feeling to the touch of the emulsifiable preparation can be made smoother. The aqueous component may contain, as a polyhydric alcohol, sorbitol, xylitol, erythritol, mannose, or trehalose, each of which is individual at ordinary temperature. Such polyhydric alcohols can impart sweetness and the like to the emulsifiable preparation because they have sweetness and the like.

A proportion of the polyhydric alcohol relative to the total amount of the alcohol is preferably 20 wt. % or greater, more preferably 50 wt. % or greater, and still more preferably 100 wt. %. When the proportion of the polyhydric alcohol relative to the total amount of the alcohol is 20 wt. % or greater, the feeling to the touch of the emulsifiable preparation can be made smoother.

When the aqueous component contains water, the content of water relative to the total weight of the emulsifiable preparation is preferably 10 wt. % or less, more preferably 8 wt. % or less, and still more preferably 5 wt. % or less. When the content of water relative to the total weight of the emulsifiable preparation is more than 10 wt. %, water may decompose the biodegradable polymer compound contained in the cellulose acetate microparticles. In contrast, when the content of water relative to the total weight of the emulsifiable preparation is 10 wt. % or less, a water-soluble component such as salt becomes soluble in the emulsifiable preparation, and it is also possible to suppress the influence of water on the biodegradable polymer compound contained in the cellulose acetate microparticles.

When the aqueous component contains an alcohol, the biodegradable polymer compound contained in the cellulose acetate microparticles can suppress the influence of the aqueous component, compared with the case where the aqueous component is only water. When the aqueous component contains an alcohol, the content of the aqueous component including the alcohol is preferably 90 wt. % or less, more preferably 70 wt. % or less, and still more preferably 60 wt. % or less relative to the total weight of the emulsifiable preparation.

### Oily Component

In the emulsifiable preparation of the present disclosure, the oily component refers to a component which is insoluble or poorly soluble in water. The oily component is a useful component that is oil-based and intended to be added to an aqueous cosmetic, a food or beverage, a pharmaceutical composition, or the like. The oily component is not particularly limited, but is preferably an oily component that is highly safe to the human body. As the oily component, for example, an oily component that can be used as an oral preparation or an oily component that is safe when applied to skin can be adopted. Examples of the oily component include oils and fats such as monoglyceride in which one fatty acid is bonded to glycerin, diglyceride in which two fatty acids are bonded to glycerin, and triglyceride in which three fatty acids are bonded to glycerin. Specific examples of the oils and fats include vegetable oils such as rapeseed oil, sesame oil, olive oil, coconut oil, camellia oil, corn oil, avocado oil, sasanqua oil, castor oil, jojoba oil, sunflower oil, and soybean oil.

In addition, examples of the oily component include esters, silicones, lactones, aldehydes, ketones, higher fatty acids, higher alcohols, essential oils, antioxidants, fat-soluble vitamins, and plant sterols. Examples of the esters include monoesters such as cetyl 2-ethylhexanoate, isopropyl myristate, cetyl octanoate, octyl palmitate, isocetyl stearate, isopropyl isostearate, octyl isopalmitate, and isopropyl sebacate; diesters such as diethyl sebacate, diisopropyl sebacate, di-2-ethylhexyl sebacate, and diisopropyl phthalate; and triesters such as glyceryl tri-2-ethylhexanoate and caprylic/capric triglyceride. Examples of the silicones include straight silicone oils such as dimethyl silicone oil, methyl phenyl silicone oil, and methyl hydrogen silicone oil, and modified silicone oils in which an organic group is introduced into a side chain or an end of the straight silicone oil. Examples of the lactones include gluconolactone, mevalonolactone, lactobionolactone, and pantolactone. Examples of the aldehydes include cinnamaldehyde and cinnamic aldehyde. Examples of the ketones include fragrances such as α-diketone.

One type of oily component may be contained, or two or more types thereof may be contained. The type and content of the oily component are appropriately selected according to the use, the solubility of the additive, or the like. The oily component is preferably from about 0.1 to about 103 parts by weight, more preferably from about 0.5 to about 10² parts by weight, and still more preferably from about 1 to about 2×10² parts by weight, relative to 100 parts by weight of the cellulose acetate microparticles. When the content of the oily component is from about 0.1 to about 103 parts by weight relative to 100 parts by weight of the cellulose acetate microparticles, an emulsifiable preparation stabilized by the cellulose acetate microparticles is prepared.

The emulsifiable preparation of the present disclosure may contain other components as long as the effects of the present disclosure are not impaired. For example, a surfactant, a thickener, a stabilizer, an antioxidant, a chelator, a preservative, a pH adjuster, a buffer, a flavoring substance, and a sweetening agent can be added to the emulsifiable preparation.

Examples of the surfactant include fatty acid soaps such as sodium laurate and sodium palmitate; anionic surfactants such as potassium lauryl sulfate, and triethanolamine alkyl sulfate ether; cationic surfactants such as stearyltrimethylammonium chloride, benzalkonium chloride, and laurylamine oxide; imidazoline-based amphoteric surfactants such as 2-cocoyl-2-imidazolinium hydroxide-1-carboxyethyloxy 2 sodium salt; betaine-based surfactants such as alkyl betaine, amide betaine, and sulfobetaine; amphoteric surfactants such as acyl methyl taurine; sorbitan fatty acid esters such as sorbitan monostearate and sorbitan sesquioleate; glycerin fatty acid esters such as glycerin monostearate; propylene glycol fatty acid esters such as propylene glycol monostearate; hydrogenated castor oil derivatives; glycerin alkyl ethers; polyoxyethylene sorbitan fatty acid esters; polyoxyethylene sorbitol fatty acid esters; polyoxyethylene glycerin fatty acid esters; polyoxyethylene fatty acid esters; polyoxyethylene alkyl ethers; polyoxyethylene alkyl phenyl ethers; Pluronic (registered trademark) types; polyoxyethylene/polyoxypropylene alkyl ethers; Tetronic types; polyoxyethylene castor oil/hydrogenated castor oil derivatives; nonionic surfactants such as sucrose fatty acid esters and alkyl glucosides; sodium dodecyl sulfate; vitamin E derivatives; and phospholipids.

The amount of the surfactant is preferably from 0.01 to 100 parts by weight, more preferably from 0.1 to 80 parts by weight, and still more preferably from 1 to 50 parts by weight relative to 100 parts by weight of the polymer compound (e.g., cellulose acetate) contained in the cellulose acetate microparticles. When the amount of the surfactant is from 0.01 to 100 parts by weight relative to 100 parts by weight of the polymer compound contained in the cellulose acetate microparticles, the emulsifiable preparation can further improve the dispersibility of the oily component.

Examples of the thickener include xanthan gum, curdlan, pullulan, guar gum derivatives, locust bean gum, carrageenan, pectin, cellulose derivatives such as hydroxyethyl cellulose and carboxymethyl cellulose, carbomers (carboxyvinyl polymers), pectin, @3-glucan, tamarind gum, polyvinylpyrrolidone, polyvinyl alcohol, polyacrylic acid, alginic acid, hyaluronic acid, and polyalkylene glycol, and salts thereof. One kind of these thickeners can be used alone or two or more kinds thereof can be used in combination. The thickener is preferably a carboxyvinyl polymer in terms of having low irritation and a high thickening effect, having low changes over time in viscosity, and having strong resistance to contamination by microorganisms. The emulsifiable preparation contains a thickener, and thus the dispersibility of the oily component and the cellulose acetate microparticles can be further stabilized.

The emulsifiable preparation of the present disclosure can be prepared, for example, by the following method. Cellulose acetate microparticles are stirred together with an aqueous component for a predetermined time. As a result, the cellulose acetate microparticles are dispersed in the aqueous component to get wet. An oily component is added to the wet cellulose acetate microparticles, and the mixture is stirred for a predetermined time. As a result, the oily component and the aqueous component are emulsified by the cellulose acetate microparticles, and thereby an emulsifiable preparation was yielded. The emulsifiable preparation may be prepared by another known method without being limited to the preparation method described above.

The emulsifiable preparation of the present disclosure is suitable for an external preparation for skin. The emulsifiable preparation of the present disclosure is excellent in storage stability and feeling to the touch, and thus can be used for aqueous cosmetics. An aqueous cosmetic can be prepared, for example, by the following method. In the emulsifiable preparation of the present disclosure, for example, an aqueous component such as water or other additives is/are added and the mixture is stirred for a predetermined time. As a result, an aqueous cosmetic is prepared in which the emulsifiable preparation supports the intended use.

The emulsifiable preparation of the present disclosure can be, for example, a sunscreen, a makeup base, a foundation, a lotion, a lipstick, a lip gloss, a hair care product, or the like. Cellulose acetate microparticles contained in the emulsifiable preparation of the present disclosure are safe for the human body. Accordingly, the emulsifiable preparation of the present disclosure can be suitably used for foods or beverages. The emulsifiable preparation of the present disclosure can be, for example, a cream, a sauce, a jelly, or a health food such as a supplement, or other beverages. In addition, the emulsifiable preparation of the present disclosure can be a pharmaceutical preparation (pharmaceutical composition) such as an oral preparation, an injection, or an external preparation such as an ointment or a poultice.

Each aspect disclosed in the present specification can be combined with any other feature disclosed herein.

### EXAMPLES

Hereinafter, the present disclosure will be specifically described with reference to examples, but the technical scope of the present disclosure is not limited by these examples. Note that each of the configurations, combinations thereof, and the like in each of the embodiments are an example, and various additions, omissions, substitutions, and other changes may be made as appropriate without departing from the spirit of the present disclosure.

### Production Example 1

First, 100 parts by weight of cellulose diacetate (available from Daicel Corporation: total degree of acetyl substitution DS=2.4) and 25 parts by weight of triacetin as a plasticizer were blended in a dry state, dried at 80° C. for 12 hours or more, and further stirred and mixed using a Henschel mixer to prepare a mixture of the cellulose acetate and the plasticizer. The resulting mixture was fed to a twin-screw extruder (PCM30 available from Ikegai Corp., a cylinder temperature of 200° C., a die temperature of 220° C.), melt-kneaded, extruded, pelletized, and a kneaded product was formed.

Then, 32 parts by weight of the pellets of the resulting kneaded product and 68 parts by weight of polyvinyl alcohol (available from The Nippon Synthetic Chemical Industry Co., Ltd., a melting point of 190° C., a saponification degree of 99.1%) as a water-soluble polymer were blended in a dry state, then fed to a twin-screw extruder (PCM30 available from Ikegai Corp., a cylinder temperature of 220° C., a die temperature of 220° C.), extruded, and a dispersion was formed.

The resulting dispersion was combined with pure water (a solvent) to give a concentration of not higher than 5 wt. % (weight of dispersion/(weight of dispersion+weight of pure water)×100), and the mixture was stirred using a Three-One Motor (BL-3000 available from Shinto Scientific Co., Ltd.) at a rotation speed of 500 rpm, at a temperature of 80° C. for 5 hours. The solution after stirring was filtered off with filter paper (No. 5A available from ADVANTEC), and the filtrate was taken out. The resulting filtrate was combined with pure water again to adjust the concentration of the dispersion to 5 wt. % or less, the mixture was further stirred at a rotation speed of 500 rpm, at a temperature of 80° C. for 5 hours, and the resultant solution was filtered off to take out the filtrate. This operation was repeated three or more times, and cellulose acetate microparticles were prepared. The average particle size of the resulting cellulose acetate microparticles was measured by the following method.

The average particle size was measured using dynamic light scattering. First, the sample was combined with pure water to adjust the concentration to approximately 100 ppm, and a pure water suspension was prepared using an ultrasonic vibrating device. Then, the particle size volume distribution was determined by laser diffraction (“Laser Diffraction/Scattering Particle Size Distribution Measuring Apparatus LA-960” available from Horiba Ltd., ultrasonic treatment for 15 minutes, a refractive index (1.500, medium (water; 1.333)), and the average particle size was measured. The average particle size (in nm, μm, etc.) herein was the value of the particle size corresponding to 50% of the integrated scattering intensity in the particle size volume distribution. The result for the average particle size of the resulting cellulose acetate microparticles is shown in Table 1.

### Production Example 2

A kneaded product was prepared in the same manner as in Production Example 1 except that the amount of triacetin was changed to 22 parts by weight; a dispersion was formed in the same manner as in Production Example 1 except that the amount of the pellets of the resulting kneaded product was changed to 34 parts by weight and the amount of polyvinyl alcohol was changed to 66 parts by weight; and cellulose acetate microparticles were prepared in the same manner as in Production Example 1 except that the resulting dispersion was combined with pure water to have a concentration of 5 wt. % or less, and the mixture was vigorously stirred at a rotation speed (200 rpm) at a temperature of 80° C. for 5 hours. The result for the average particle size of the resulting cellulose acetate microparticles is shown in Table 1.

### Production Example 3

A kneaded product was prepared in the same manner as in Production Example 1 except that triacetin was replaced with acetyl triethyl citrate as the plasticizer; a dispersion was formed in the same manner as in Production Example 1 except that the amount of the pellets of the resulting kneaded product was changed to 14 parts by weight and the amount of polyvinyl alcohol was changed to 86 parts by weight; and cellulose acetate microparticles were prepared in the same manner as in Production Example 1 except that the resulting dispersion was combined with pure water to have a concentration of 5 wt. % or less, and the mixture was vigorously stirred at a rotation speed (100 rpm) at a temperature of 80° C. for 3 hours. The result for the average particle size of the resulting cellulose acetate microparticles is shown in Table 1.

### Example 1

As shown in Table 1 below, 2 parts by weight of cellulose acetate microparticles (average particle size: 5 μm, available from Daicel Corporation) of Production Example 1 as microparticles, 5 parts by weight of 1,3-butylene glycol (available from Daicel Corporation) as an aqueous component, 3 parts by weight of dipropylene glycol (available from ADEKA Corporation) as an aqueous component, 5 parts by weight of cetyl 2-ethylhexanoate (available from Kokyu Alcohol Kogyo Co., Ltd.) as an oily component, 64 parts by weight of pure water as a solvent, 20 parts by weight of a 1 wt. % carbomer solution (AQUPEC 705E, available from Sumitomo Seika Chemicals Company, Limited), and 1 part by weight of a 10 wt. % potassium hydroxide solution as a solvent were prepared.

The prepared cellulose acetate microparticles and the aqueous component were stirred at ordinary temperature for 3 minutes using a mill: Disper (available from PRIMIX Corporation), and then the resulting mixed liquid was left to stand still for 10 minutes to wet the cellulose acetate microparticles with the aqueous component. The oily component was added to the mixed liquid after wetting, and the mixture was stirred at ordinary temperature for 10 minutes using the mill: Disper (available from PRIMIX Corporation), and thereby an emulsifiable preparation was yielded. Subsequently, a solvent was added to the resulting emulsifiable preparation, and the mixture was stirred at ordinary temperature for 10 minutes using the mill: Disper (available from PRIMIX Corporation), and thereby an aqueous cosmetic was yielded.

### Example 2 and Comparative Examples 1 and 2

An aqueous cosmetic was prepared in the same manner as in Example 1 except that cellulose acetate microparticles were changed as shown in Table 1 below. In Example 2, the cellulose acetate microparticles prepared in Production Example 2 were used as microparticles. In Comparative Example 1, silica particles (Sunsphere L-51, average particle size: 5 μm, available from AGC Si-Tech Co., Ltd.) were used as microparticles. Further, in Comparative Example 2, the cellulose acetate microparticles prepared in Production Example 3 were used as microparticles.

### Evaluation

The dispersion stability and the touch feeling to the skin of the aqueous cosmetics prepared in Examples 1 and 2 and Comparative Examples 1 and 2 were evaluated by the following methods.

### Dispersion Stability Evaluation

20 mL of the resulting aqueous cosmetic was placed in a screw bottle and left to stand still for 3 months under conditions at 25° C. and 75% RH, followed by visual observation of whether or not the aqueous cosmetic was uniform. Then, the dispersion stability was evaluated in the following criteria.

Evaluation Criteria

Uniform: Good

Ununiform: Poor

### Touch Feeling to the Skin

10 monitors applied 1 g of the aqueous cosmetic to their skins, and evaluated whether or not the aqueous cosmetic was smooth to the touch according to the following criteria.

### Evaluation Criteria

8 or more monitors answered that the aqueous cosmetic was smooth to the touch: Good

3 to 7 monitors answered that the aqueous cosmetic was smooth to the touch: Marginal

2 or less monitors answered that the aqueous cosmetic was smooth to the touch: Poor

The results are summarized and shown in Table 1 below.

TABLE 1

| | | | | Comparative | Comparative |
|---|---|---|---|---|---|
| | Component | Example 1 | Example 2 | Example 1 | Example 2 |
| Microparticles | Cellulose acetate | 2 | | | |
| | microparticles | | | | |
| | (average particle size: 5 μm) | | | | |
| | Cellulose acetate | | 2 | | |
| | microparticles | | | | |
| | (average particle size: 7 μm) | | | | |
| | Silica particles | | | 2 | |
| | (average particle size: 5 μm) | | | | |
| | Cellulose acetate | | | | 2 |
| | microparticles | | | | |
| | (average particle size: 0.3 μm) | | | | |
| Aqueous | 1,3-butylene glycol | 5 | 5 | 5 | 5 |
| component | Dipropylene glycol | 3 | 3 | 3 | 3 |
| Oily | Cetyl ethylhexanoate | 5 | 5 | 5 | 5 |
| component | | | | | |
| Solvent | Water | 64 | 64 | 64 | 64 |
| | 1 wt. % carbomer solution | 20 | 20 | 20 | 20 |
| | 10% KOH solution | 1 | 1 | 1 | 1 |
| Evaluation | Dispersion stability | Good | Good | Good | Good |
| | Feeling to the touch | Good | Good | Marginal | Poor |

### Example A-1

First, 100 parts by weight of cellulose diacetate (available from Daicel Corporation: total degree of acetyl substitution DS=2.4, SP value: 24 (MPa^{1/2})) and 25 parts by weight of triacetin as a plasticizer were blended in a dry state, dried at 80° C. for 12 hours or longer, further stirred and mixed using a Henschel mixer, and a mixture of the cellulose acetate and the plasticizer was prepared. The resulting mixture was fed to a twin-screw extruder (PCM30 available from Ikegai Corp., a cylinder temperature of 200° C., a die temperature of 220° C.), melt-kneaded, extruded, pelletized, and a kneaded product was formed.

100 parts by weight of the pellets of the resulting kneaded product, 271 parts by weight of polyvinyl alcohol (PVA available from The Nippon Synthetic Chemical Industry Co., Ltd.: melting point: 190° C., degree of saponification: 99.1%, SP value: 34 (MPa^{1/2})) as the first thermoplastic polymer, and 21 parts by weight of polyethylene glycol (PEG, SP value: 20 (MPa^{1/2})) as the second thermoplastic polymer were dry-blended. Then, the mixture was fed to a twin-screw extruder (PCM30 available from Ikegai Corp., cylinder temperature: 220° C., die temperature: 220° C.), and extruded to form a mixture of cellulose acetate.

The resulting mixture of cellulose acetate was combined with pure water (a solvent) to give a concentration of not more than 5 wt. % (weight of mixture/(weight of mixture+weight of pure water)×100), and the mixture was stirred using a Three-One Motor (BL-3000 available from Shinto Scientific Co., Ltd.) at a rotation speed of 100 rpm, at a temperature of 80° C. for 3 hours. The solution after stirring was filtered off with filter paper (No. 5A available from ADVANTEC), and the filtrate was taken out. The resulting filtrate was prepared using pure water again to give a concentration of the mixture of not more than 5 wt. %, the mixture was further stirred at a rotation speed of 100 rpm, at a temperature of 80° C. for 3 hours, and the solution was filtered off to take out the filtrate. This operation was repeated three or more times, and cellulose acetate particles were obtained.

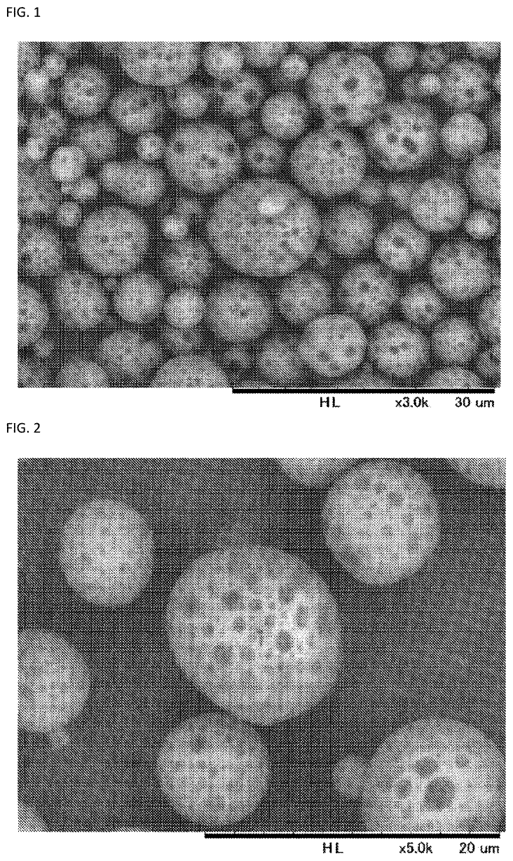

The average particle size, coefficient of variation of particle size, sphericity, oil absorption, degree of surface smoothness, bulk specific gravity, and RSSA of the resulting cellulose acetate particles were determined, and the biodegradability and tactile sensation were evaluated. The results are shown in Table 1. Note that the measurement or evaluation of the average particle size, coefficient of variation of particle size, sphericity, oil absorption, degree of surface smoothness, bulk specific gravity, RSSA, biodegradability, and tactile sensation were measured or evaluated by the following methods. The scanning electron microscope (SEM) image is as shown in FIG. 1-3. Lengths of scale bars in FIGS. 1 to 3 are 30 μm in the SEM image magnified 3000 times, 20 μm in the SEM image magnified 5000 times, and 5.00 μm in the SEM image magnified 6000 times, respectively.

### Average Particle Size and Coefficient of Variation of Particle Size

The average particle size was measured using dynamic light scattering. First, the sample was combined with pure water to adjust the concentration to approximately 100 ppm, and a pure water suspension was prepared using an ultrasonic vibrating device. Then, the particle size volume distribution was determined by laser diffraction (“Laser Diffraction/Scattering Particle Size Distribution Measuring Device LA-960” available from Horiba Ltd., ultrasonic treatment for 15 minutes, a refractive index (1.500, medium (water; 1.333)), and the average particle size was measured. The average particle size (in nm, μm, etc.) herein was the value of the particle size corresponding to 50% of the integrated scattering intensity in the particle size volume distribution. In addition, the coefficient of variation (%) of the particle size was calculated by an equation: standard deviation of particle size/average particle size×100.

### Sphericity

Using an image of particles observed with a scanning electron microscope (SEM), the major axis length and the minor axis length of 30 randomly selected particles were measured to determine the (minor axis length)/(major axis length) ratio of each particle, and the average value of the (minor axis length)/(major axis length) ratios was taken as the sphericity.

### Oil Absorption

The oil absorption was determined by Test methods for pigments, specified in JIS K 5101-13-1: 2004 (ISO 787-5: 1980)—Part 13: Oil absorption—Section 1: Refined linseed oil method.

### Degree of Surface Smoothness

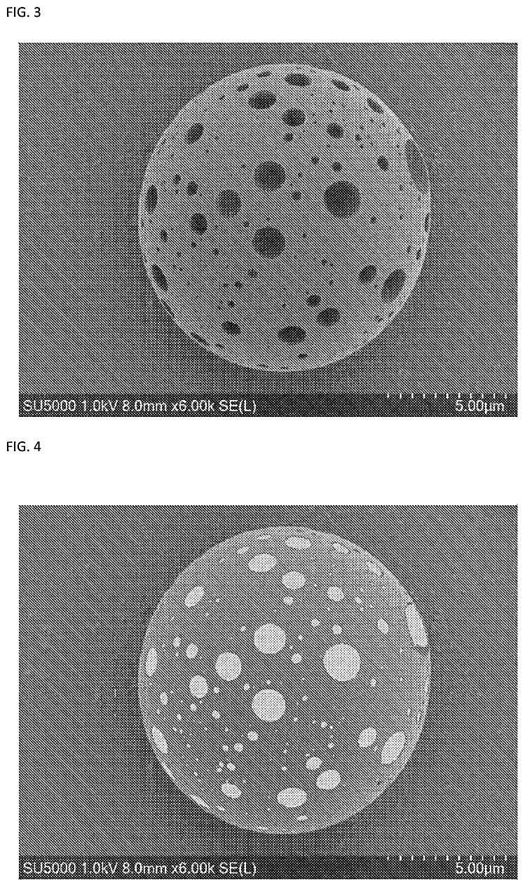

A scanning electron micrograph of the particles was taken at a magnification of 2500 to 6000 times (see FIG. 3 for an example of a micrograph of the cellulose acetate particles in Example A-1, taken with “SU 5000”: trade name, available from Hitachi High-Technologies Corporation), and the image was binarized using an image processor WinrOOF (available from Mitani Corporation) (see FIG. 4 for the binarized image of the micrograph of FIG. 3). A region including the center and/or near the center of one particle was randomly selected from the binarized image. The area ratio of a portion (shade portion) corresponding to a recess of unevenness in the region was calculated, and the degree of surface smoothness (%) of one of the particles was calculated by the following formula:

Degree of surface smoothness of one particle (%)=(1−area ratio of recesses)×100

Area ratio of recesses=area of recessed portions in the any area/the any area

The average value of the degree of surface smoothness of randomly selected 10 particle samples, that is, from n1 to 10, was taken as the degree of surface smoothness (%). The higher this numerical value, the higher the degree of surface smoothness is. Note that the region used for calculating the area ratio may be any areas each smaller than the particle including the center and/or near the center of one particle. In addition, the size of the area may be 5 μm square for the particle with a diameter of 15 μm.

### Bulk Specific Gravity

The bulk specific gravity was measured according to “JIS K 1201-1”.

### Relative Specific Surface Area: RSSA

A specific surface area calculated from the measurement results of the particle size distribution, assuming that the particle was a truly spherical particle having a smooth surface, was defined as “theoretical specific surface area”, and the specific surface area measured by the BET method was defined as “specific surface area measurement value”, the following relationship was established: relative specific surface area (RSSA)=specific surface area measurement value/theoretical specific surface area.

The method of measuring the specific surface area by the BET method is as follows. The specific surface area using the BET specific surface area measurement by nitrogen adsorption can be determined by previously evacuating ports holding samples in a MasterPrep degasser (available from Quantachrome Instruments) under heating at a temperature of 100° C. for about 1 hour, then measuring nitrogen adsorption at about 7 points in a relative pressure range of 0.05 to 0.28 by a nitrogen gas adsorption method using a specific surface area measuring device (“Autosorb iQStation 2” available from Quantachrome Instruments), and calculating the specific surface areas using the BET method.

### Biodegradability

Biodegradability was evaluated by biodegradation rate. The biodegradation rate was measured by a method using activated sludge in accordance with JIS K6950. The activated sludge was obtained from a municipal sewage-treatment plant. About 300 mL of a supernatant (activated sludge concentration: about 360 ppm) obtained by allowing the activated sludge to stand for approximately 1 hour was used per culture bottle. The measurement was started when 30 mg of the sample was stirred in the supernatant, and then the sample was measured every 24 hours until after 720 hours, that is until after 30 days, a total of 31 times. Details of the measurement are as follows. The biochemical oxygen demand (BOD) in each culture bottle was measured using a Coulometer OM3001 available from Ohkura Electric Co., Ltd. The percentage of the biochemical oxygen demand (BOD) to the theoretical biochemical oxygen demand (BOD) in complete degradation based on the chemical composition of each sample was taken as the biodegradation rate (wt. %), and the biodegradability was evaluated as follows.

Excellent: more than 60 wt. %. Good: 40 wt. % or greater and 60 wt. % or less.
Marginal: 10 wt. % or greater and less than 40 wt. %. Poor: less than 10 wt. %.

### Tactile Sensation

Sensory evaluation was performed according to a panel test by 20 panelists for the tactile sensation of the particles. Panelists were instructed to touch the particles to evaluate comprehensively softness, smoothness and moist feeling, on a scale with a maximum score of 5 points according to the following criteria, and an average score from 20 panelists was calculated.

Good: 5. Slightly good: 4. Average: 3. Slightly poor: 2. Poor: 1.

### Scanning Electron Microscope (SEM) Image

Scanning electron microscope (SEM) images were taken at magnifications of 3000 times, 5000 times, and 6000 times. A scanning electron microscope (trade name “TM 3000” available from Hitachi High-Technologies Corporation) was used to take images at magnifications of 3000 times and 5000 times, and a scanning electron microscope (trade name “SU 5000” available from Hitachi High-Technologies Corporation) was used to take images at a magnification of 6000 times.

### Examples A-2, A-3, A-5, and A-7 to A-12

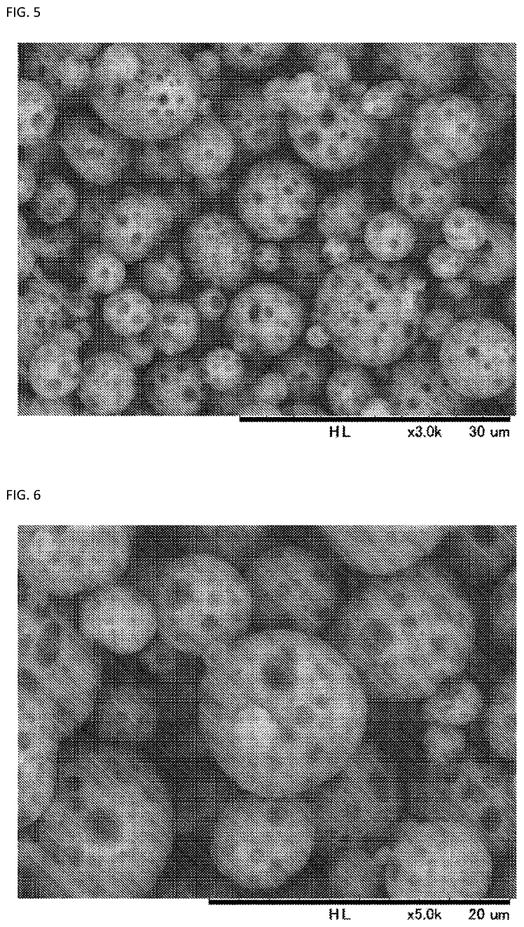

Cellulose acetate particles were prepared in the same manner as in Example A-1 except that the type and blending amount of each of the plasticizer, the first thermoplastic resin, and the second thermoplastic resin were changed as shown in Table 1. Each physical property of the resulting cellulose acetate particles was evaluated according to the measurement methods described above. The results are shown in Table 1. In addition, the scanning electron microscope (SEM) images of Example A-1 are shown in FIG. 1 (magnification: 3000), FIG. 2 (magnification: 5000), and FIG. 3 (magnification: 6000), respectively. The SEM images of Example A-12 are shown in FIG. 5 (magnification: 3000) and FIG. 6 (magnification: 5000), respectively.

### Examples A-4 and A-6

In Example A-4, the cellulose acetate was replaced with cellulose diacetate (available from Daicel Corporation, total degree of acetyl substitution DS=2.8, SP value: 22.6 (MPa^{1/2})). In Example A-6, the cellulose acetate was replaced with cellulose diacetate (available from Daicel Corporation, total degree of acetyl substitution DS=1.8, SP value: 26 (MPa^{1/2})). Further, cellulose acetate particles were prepared in the same manner as in Example A-1 except that the type and blending amount of each of the plasticizer, the first thermoplastic resin, and the second thermoplastic resin were changed as shown in Table 1. Each physical property of the resulting cellulose acetate particles was evaluated according to the measurement methods described above. The results are shown in Table 1.

### Comparative Examples A-1 to A-3, A-5, A-7 to A-11

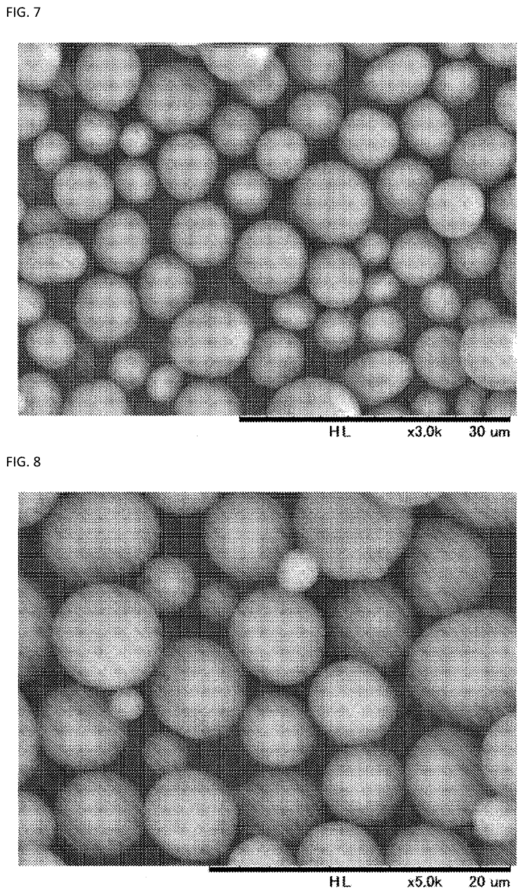

Cellulose acetate particles were prepared in the same manner as in Example A-1 except that the type and blending amount of each of the plasticizer and the first thermoplastic resin were changed as shown in Table 2, and the second thermoplastic resin was not added. Each physical property of the resulting cellulose acetate particles was evaluated according to the measurement methods described above. The results are shown in Table 2. The scanning electron microscope (SEM) images of Comparative Example A-1 are shown in FIG. 7 (3000 times) and FIG. 8 (5000 times).

### Comparative Example A-4 and Comparative Example A-6

Cellulose acetate particles of Comparative Example A-4 were prepared in the same manner as in Example A-1 except that the cellulose acetate was replaced with cellulose diacetate (available from Daicel Corporation, total degree of acetyl substitution DS=2.8, SP value: 22.6 (MPa^{1/2})), the type and blending amount of each of the plasticizer and the first thermoplastic resin were changed as shown in Table 2, and the second thermoplastic resin was not added. Cellulose acetate particles of Comparative Example A-6 were prepared in the same manner as in Example A-1 except that the cellulose acetate was replaced with cellulose diacetate (available from Daicel Corporation, total degree of acetyl substitution DS=1.8, SP value: 26 (MPa^{1/2})), the type and blending amount of each of the plasticizer and the first thermoplastic resin were changed as shown in Table 2, and the second thermoplastic resin was not added. Each physical property of the resulting cellulose acetate particles was evaluated according to the measurement methods described above. The results are shown in Table 2.

### Comparative Examples A-12 to 15

Cellulose acetate particles were prepared in the same manner as in Example A-1 except that the type and blending amount of each of the first thermoplastic resin and the second thermoplastic resin were changed as shown in Table 3. Each physical property of the resulting cellulose acetate particles was evaluated according to the measurement methods described above. The results are shown in Table 3.

TABLE 1

| | | Example | Example | Example | Example | Example | Example | Example |
|---|---|---|---|---|---|---|---|---|
| | | A-1 | A-2 | A-3 | A-4 | A-5 | A-6 | A-7 |
| Total degree of acetyl | | 2.4 | 2.4 | 2.4 | 2.8 | 2.4 | 1.8 | 2.4 |
| substitution (DS) | | | | | | | | |
| Plasticizer | Type | Triacetin | Acetyl | Triacetin | Diethyl | Acetyl | Diacetin | Triacetin |
| | | | triethyl | | phthalate | triethyl | | |
| | | | citrate | | | citrate | | |
| | Blending | 25 | 25 | 25 | 25 | 25 | 25 | 20 |
| | amount (part | | | | | | | |
| | by weight) | | | | | | | |
| First | Type | PVA | PVA | PVA | PVA | PVA | PVA | PVA |
| thermoplastic | | | | | | | | |
| resin | Blending | 271 | 271 | 271 | 271 | 271 | 271 | 261 |
| | amount (part | | | | | | | |
| | by weight) | | | | | | | |
| Second | Type | PEG | PEG | PEG | PEG | PEG | PEG | PEG |
| thermoplastic | Blending | 21 | 21 | 21 | 21 | 21 | 21 | 20 |
| resin | amount (part | | | | | | | |
| | by weight) | | | | | | | |
| Cellulose | Average | 13.3 μm | 210 nm | 20 μm | 27 μm | 7.2 μm | 10.1 μm | 40 μm |
| acetate | particle size | | | | | | | |
| particles | Coefficient of | 36% | 40% | 38% | 40% | 37% | 39% | 38% |
| | variation of | | | | | | | |
| | particle size | | | | | | | |
| | Sphericity | 0.95 | 0.94 | 0.96 | 0.95 | 0.96 | 0.96 | 0.96 |
| | Oil absorption | 72 | 86 | 70 | 68 | 77 | 75 | 66 |
| | (ml/100 g) | | | | | | | |
| | Degree of | 80% | 75% | 85% | 83% | 86% | 87% | 78% |
| | surface | | | | | | | |
| | smoothness | | | | | | | |
| | bulk specific | 0.50 | 0.43 | 0.46 | 0.43 | 0.47 | 0.49 | 0.50 |
| | gravity | | | | | | | |
| | Theoretical | 0.35 | 0.03 | 0.24 | 0.18 | 0.64 | 0.46 | 0.12 |
| | specific surface | | | | | | | |
| | area (m²/g) | | | | | | | |
| | BET specific | 3.53 | 0.30 | 1.89 | 2.23 | 7.26 | 5.47 | 1.24 |
| | surface area | | | | | | | |
| | (m²/g) | | | | | | | |
| | RSSA | 10 | 11.2 | 7.9 | 12.4 | 11.4 | 11.9 | 10 |
| | Biodegradability | Excellent | Excellent | Excellent | Marginal | Excellent | Excellent | Excellent |
| | Tactile | 4.4 | 4 | 4.2 | 3.9 | 4.2 | 4.4 | 4 |
| | sensation | | | | | | | |

| | | Example | Example | Example | Example | Example |
|---|---|---|---|---|---|---|
| | | A-8 | A-9 | A-10 | A-11 | A-12 |
| Total degree of acetyl | | 2.4 | 2.4 | 2.4 | 2.4 | 2.4 |
| substitution (DS) | | | | | | |
| Plasticizer | Type | Triacetin | Triacetin | Triacetin | Triacetin | Triacetin |
| | Blending | 25 | 25 | 25 | 25 | 25 |
| | amount (part | | | | | |
| | by weight) | | | | | |
| First | Type | PVA | PVA | PVA | Thermoplastic | PVA |
| thermoplastic | | | | | starch | |
| resin | Blending | 271 | 271 | 271 | 271 | 283 |
| | amount (part | | | | | |
| | by weight) | | | | | |
| Second | Type | PEG | PEG | PEG | PEG | PEG |
| thermoplastic | Blending | 21 | 21 | 21 | 21 | 8 |
| resin | amount (part | | | | | |
| | by weight) | | | | | |
| Cellulose | Average | 13.8 μm | 11.2 μm | 14.1 μm | 13.8 μm | 10.9 μm |
| acetate | particle size | | | | | |
| particles | Coefficient of | 44% | 36% | 37% | 39% | 34% |
| | variation of | | | | | |
| | particle size | | | | | |
| | Sphericity | 0.83 | 0.98 | 0.97 | 0.95 | 0.96 |
| | Oil absorption | 71 | 73 | 71 | 72 | 74 |
| | (ml/100 g) | | | | | |
| | Degree of | 80% | 82% | 85% | 83% | 80% |
| | surface | | | | | |
| | smoothness | | | | | |
| | bulk specific | 0.48 | 0.51 | 0.46 | 0.48 | 0.50 |
| | gravity | | | | | |
| | Theoretical | 0.34 | 0.42 | 0.33 | 0.34 | 0.43 |
| | specific surface | | | | | |
| | area (m²/g) | | | | | |
| | BET specific | 2.66 | 3.62 | 3.10 | 3.03 | 5.64 |
| | surface area | | | | | |
| | (m²/g) | | | | | |
| | RSSA | 7.8 | 8.7 | 9.3 | 8.9 | 13.2 |
| | Biodegradability | Excellent | Excellent | Excellent | Excellent | Excellent |
| | Tactile | 3.9 | 4.2 | 4.4 | 4.1 | 4.5 |
| | sensation | | | | | |

TABLE 2

| | | Compar- | Compar- | Compar- | Compar- | Compar- | Compar- |
|---|---|---|---|---|---|---|---|
| | | ative | ative | ative | ative | ative | ative |
| | | Example | Example | Example | Example | Example | Example |
| | | A-1 | A-2 | A-3 | A-4 | A-5 | A-6 |
| Total degree of acetyl | | 2.4 | 2.4 | 2.4 | 2.8 | 2.4 | 1.8 |
| substitution (DS) | | | | | | | |
| Plasticizer | Type | Triacetin | Acetyl | Triacetin | Diethyl | Acetyl | Diacetin |
| | | | triethyl | | phthalate | triethyl | |
| | | | citrate | | | citrate | |
| | Blending | 25 | 25 | 25 | 25 | 25 | 25 |
| | amount (part | | | | | | |
| | by weight) | | | | | | |
| First | Type | PVA | PVA | PVA | PVA | PVA | PVA |
| thermoplastic | | | | | | | |
| resin | Blending | 292 | 292 | 292 | 292 | 292 | 292 |
| | amount (part | | | | | | |
| | by weight) | | | | | | |
| Second | Type | — | — | — | — | — | — |
| thermoplastic | Blending | — | — | — | — | — | — |
| resin | amount (part | | | | | | |
| | by weight) | | | | | | |
| Cellulose | Average | 4.2 μm | 100 nm | 14 μm | 20 μm | 1.1 μm | 2.6 μm |
| acetate | particle size | | | | | | |
| particles | Coefficient of | 38% | 42% | 41% | 40% | 37% | 39% |
| | variation of | | | | | | |
| | particle size | | | | | | |
| | Sphericity | 0.98 | 0.94 | 0.97 | 0.95 | 0.97 | 0.97 |
| | Oil absorption | 51 | 56 | 45 | 43 | 55 | 53 |
| | (ml/100 g) | | | | | | |
| | Degree of | 100% | 99% | 100% | 100% | 99% | 99% |
| | surface | | | | | | |
| | smoothness | | | | | | |
| | bulk specific | 0.63 | 0.62 | 0.61 | 0.62 | 0.62 | 0.61 |
| | gravity | | | | | | |
| | Theoretical | 1.09 | 48.10 | 0.35 | 0.25 | 3.86 | 1.73 |
| | specific surface | | | | | | |
| | area (m²/g) | | | | | | |
| | BET specific | 2.84 | 91.39 | 0.84 | 0.51 | 5.79 | 2.77 |
| | surface area | | | | | | |
| | (m²/g) | | | | | | |
| | RSSA | 2.6 | 1.9 | 2.4 | 2 | 1.5 | 1.6 |
| | Biodegradability | Excellent | Excellent | Excellent | Marginal | Excellent | Excellent |
| | Tactile | 3.2 | 2.7 | 3 | 2.8 | 2.6 | 3 |
| | sensation | | | | | | |

| | | Compar- | Compar- | Compar- | Compar- | Compar- |
|---|---|---|---|---|---|---|
| | | ative | ative | ative | ative | ative |
| | | Example | Example | Example | Example | Example |
| | | A-7 | A-8 | A-9 | A-10 | A-11 |
| Total degree of acetyl | | 2.4 | 2.4 | 2.4 | 2.4 | 2.4 |
| substitution (DS) | | | | | | |
| Plasticizer | Type | Triacetin | Triacetin | Triacetin | Triacetin | Triacetin |
| | Blending | 20 | 25 | 25 | 25 | 25 |
| | amount (part | | | | | |
| | by weight) | | | | | |
| First | Type | PVA | PVA | PVA | PVA | Thermoplastic |
| thermoplastic | | | | | | starch |
| resin | Blending | 281 | 292 | 292 | 292 | 292 |
| | amount (part | | | | | |
| | by weight) | | | | | |
| Second | Type | — | — | — | — | — |
| thermoplastic | Blending | — | — | — | — | — |
| resin | amount (part | | | | | |
| | by weight) | | | | | |
| Cellulose | Average | 32 μm | 4.8 μm | 5.2 μm | 7.1 μm | 6.8 μm |
| acetate | particle size | | | | | |
| particles | Coefficient of | 38% | 44% | 36% | 37% | 39% |
| | variation of | | | | | |
| | particle size | | | | | |
| | Sphericity | 0.96 | 0.83 | 0.98 | 0.97 | 0.95 |
| | Oil absorption | 43 | 51 | 51 | 49 | 49 |
| | (ml/100 g) | | | | | |
| | Degree of | 100% | 100% | 100% | 100% | 100% |
| | surface | | | | | |
| | smoothness | | | | | |
| | bulk specific | 0.63 | 0.6 | 0.6 | 0.58 | 0.58 |
| | gravity | | | | | |
| | Theoretical | 0.17 | 0.96 | 0.89 | 0.66 | 0.69 |
| | specific surface | | | | | |
| | area (m²/g) | | | | | |
| | BET specific | 0.35 | 2.78 | 2.40 | 0.99 | 1.24 |
| | surface area | | | | | |
| | (m²/g) | | | | | |
| | RSSA | 2.1 | 2.9 | 2.7 | 1.5 | 1.8 |
| | Biodegradability | Excellent | Excellent | Excellent | Excellent | Excellent |
| | Tactile | 2.6 | 2.7 | 3.1 | 3 | 3.1 |
| | sensation | | | | | |

TABLE 3

| | | Comparative | Comparative | Comparative | Comparative |
|---|---|---|---|---|---|
| | | Example | Example | Example | Example |
| | | A-12 | A-13 | A-14 | A-15 |
| Total degree of acetyl | | 2.4 | 2.4 | 2.4 | 2.4 |
| substitution (DS) | | | | | |
| Plasticizer | Type | Triacetin | Triacetin | Triacetin | Triacetin |
| | Blending | 25 | 25 | 25 | 25 |
| | amount (part by | | | | |
| | weight) | | | | |
| First | Type | PVA | PVA | PVA | PVA |
| thermoplastic | Blending | 125 | 125 | 125 | 188 |
| resin | amount (part by | | | | |
| | weight) | | | | |
| Second | Type | Ethylene | Propylene | 1,3-butylene | Glycerin |
| thermoplastic | | glycol | glycol | glycol | |
| resin | Blending | 25 | 25 | 25 | 21 |
| | amount (part by | | | | |
| | weight) | | | | |
| Cellulose | Average particle | 10.9 μm | 8.5 μm | 6.9 μm | 6.2 μm |
| acetate | size | | | | |
| particles | Coefficient of | 44% | 135% | 41% | 35% |
| | variation of | | | | |
| | particle size | | | | |
| | Sphericity | 0.93 | 0.84 | 0.93 | 0.95 |
| | Oil absorption | 47 | 48 | 49 | 50 |
| | (ml/100 g) | | | | |
| | Degree of | 99% | 99% | 100% | 100% |
| | surface | | | | |
| | smoothness | | | | |
| | bulk specific | 0.61 | 0.61 | 0.62 | 0.62 |
| | gravity | | | | |
| | Theoretical | 0.45 | 0.63 | 0.71 | 0.75 |
| | specific surface | | | | |
| | area (m²/g) | | | | |
| | BET specific | 0.90 | 1.13 | 1.63 | 1.05 |
| | surface area | | | | |
| | (m²/g) | | | | |
| | RSSA | 2 | 1.8 | 2.3 | 1.4 |
| | Biodegradability | Excellent | Excellent | Excellent | Excellent |
| | Tactile | 2.9 | 3 | 3.2 | 3.1 |
| | sensation | | | | |

As shown in Table 1-3, the cellulose acetate particles in Examples have excellent biodegradability, excellent tactile sensation, particularly soft tactile sensation, and excellent oil absorbability.

### Example B-1

### Preparation of Liquid Foundation

Components shown in Table 4 were mixed, then stirred well, and the mixture was filled into a container to prepare liquid foundation. The tactile sensation of the resulting liquid foundation was evaluated by the method described below. The results are shown in Table 12.

TABLE 4

| Component | Product name, etc. | wt. % |
|---|---|---|
| Cyclopentasiloxane | KF-995 (Shin-Etsu Chemical Co., Ltd.) | 15.2 |
| Mineral oil | HICALL K-230 (KANEDA Co., Ltd.) | 5.0 |
| Ethylhexyl methoxycinnamate | Uvinul MC80 (BASF) | 4.0 |
| Isononyl isononanoate | KAK-99 (Kokyu Alcohol Kogyo Co., Ltd.) | 3.0 |
| Disteardimonium hectorite, | Bentone Gel VS-5 PC V HV (Elementis) | 3.0 |
| cyclopentasiloxane, other | | |
| Phytosteryl macadamiate | Plandool-MAS (Nippon Fine Chemical | 0.3 |
| | Co., Ltd.) | |
| Trimethylsiloxysilicate, | MQ-1640 Flake Resin (Dow Corning | 0.3 |
| polypropylsilsesquioxane | Toray Co., Ltd.) | |
| PEG-10 dimethicone | KF-6017P (Shin-Etsu Chemical Co., Ltd.) | 1.5 |
| Polyglyceryl oleate-2, | PolyAquol OS2 (innovacos) | 1.0 |
| polyhydroxystearic acid, | | |
| polyglyceryl stearate-2 | | |
| Titanium oxide, | SDL-Ti70 (Daito Kasei Kogyo Co., Ltd.) | 12.3 |
| cyclopentasiloxane, other | | |
| Iron oxide, | SDL-IOY50 (Daito Kasei Kogyo Co., Ltd.) | |
| cyclopentasiloxane, | SDL-IOR50 (Daito Kasei Kogyo Co., Ltd.) | 3.0 |
| other | SDL-IOB50 (Daito Kasei Kogyo Co., Ltd.) | |
| Example A-1: cellulose acetate particles | | 3.0 |
| BG | 1,3-BG (UK) (Daicel Corporation) | 6.0 |
| Phenoxyethanol | Phenoxyethanol-SP (Yokkaichi Chemical | 0.3 |
| | Co., Ltd.) | |

| | |
|---|---|
| Sodium chloride | 1.0 |
| EDTA-2Na | 0.03 |
| Purified water | Remaining amount |
| Total | 100.0 |

### Tactile Sensation

Sensory evaluation was performed according to a panel test by 20 panelists for the tactile sensation of the compositions prepared by blending the particles. Panelists were instructed to use the compositions to evaluate comprehensively both smoothness and moist feeling, on a scale with a maximum score of 5 points according to the following criteria, and an average score from 20 panelists was calculated.

Good: 5. Slightly good: 4. Average: 3. Slightly poor: 2. Poor: 1.

### Example B-2

### Preparation of Sunscreen

Components shown in Table 5 were mixed, then stirred well, and the mixture was filled into a container to prepare a sunscreen. The tactile sensation of the resulting sunscreen was evaluated by the method described above. The results are shown in Table 12.

TABLE 5

| Component | Product name, etc. | wt. % |
|---|---|---|
| Diethylamino hydroxybenzoyl hexyl benzoate | Uvinul A Plus Glanular(BASF) | 2.00 |
| Bis-ethylhexyloxyphenol methoxyphenyl | Tnosorb S(BASF) | 0.50 |
| triazine | | |
| Ethylhexyl methoxycinnamate, BHT | Uvinul MC80 (BASF) | 7.00 |
| Diisopropyl sebacate | IPSE (Nippon Fine Chemical Co., | 10.00 |
| | Ltd.) | |
| Dimethicone | KF-96A-10CS (Shin-Etsu | 2.00 |
| | Chemical Co., Ltd.) | |
| Isododecane | Marukasol R (Maruzen | 26.47 |
| | Petrochemical Co., Ltd.) | |
| Trimethylsiloxysilicate | MQ-1640 Flake Resin (Dow | 1.00 |
| | Corning Toray Co., Ltd.) | |
| PEG-9 polydimethylsiloxyethyl | KF-6028 (Shin-Etsu Chemical Co., | 2.00 |
| dimethicone | Ltd.) | |
| Titanium oxide, etc. | DIS-OP-10A (Sakai Chemical | 4.00 |
| | Industry Co., Ltd.) | |
| Zinc oxide, etc. | DIF-OP-3W (Sakai Chemical | 10.00 |
| | Industry Co., Ltd.) | |
| Example A-1: cellulose acetate particles | | 5.00 |
| Purified water | | 19.30 |
| BG | 1,3-BG (UK) (Daicel Corporation) | 3.00 |
| Phenoxyethanol | Phenoxyethanol-SP (Yokkaichi | 0.20 |
| | Chemical Co., Ltd.) | |
| Ethanol | | 7.00 |
| Sodium chloride | | 0.50 |
| EDTA-2Na | | 0.03 |
| Total | | 100.0 |

### Example B-3

### Preparation of Powder Foundation

Components A shown in Table 6 were roughly mixed, components B which had been uniformly dissolved were added thereto, and the resultant mixture was stirred well. Then, the mixture was filled into a container to prepare powder foundation. The tactile sensation of the resulting powder foundation was evaluated by the method described above. The results are shown in Table 12.

TABLE 6

| Component | wt. % |
|---|---|
| (Component (A)) | |
| Example A-1: cellulose acetate particles | 7.50 |
| SI 01-2 talc JA-46R | 29.67 |
| Mica Y-2300 | 20.00 |
| SI01-2 sericite FSE | 33.00 |
| SI 01-2 titanium oxide CR-50 | 6.50 |
| SI-2 yellow iron oxide LLXLO | 2.30 |
| SI-2 red iron oxide RED R-516L | 0.59 |
| SI-2 black iron oxide BL-100 | 0.44 |
| Component (A) total | 100.00 |
| (Component B) | |
| Dimethicone (20) | 20.00 |
| Dimethicone (350) | 20.00 |
| Glyceryl isostearate | 7.20 |
| Triethylhexanoin | 17.00 |
| Octyldodecyl oleate | 31.55 |
| Sorbitan stearate | 1.00 |
| Polyglyceryl-2 oleate | 3.10 |
| Propylparaben | 0.10 |
| Tocopherol | 0.05 |
| Component (B) total | 100.0 |
| (Final blending) | |
| Component (A) | 90.00 |
| Component (B) | 100.00 |

### Example B-4

### Preparation of Makeup Base

A component C shown in Table 7 was dispersed in components A, and the mixture was stirred well. Components B were added thereto, and the resultant mixture was stirred well. Then, the mixture was filled into a container to prepare a makeup base. The tactile sensation of the resulting makeup base was evaluated by the method described above. The results are shown in Table 12.

TABLE 7

| Component | wt. % |
|---|---|
| (Component (A)) | |
| (Dimethicone/(PEG-10/5) crosspolymer, dimethicone | 3.50 |
| PEG-9 polydimethylsiloxyethyl dimethicone | 2.00 |
| Dimethicone | 5.00 |
| Isononyl isononanoate | 4.50 |
| Octyl methoxycinnamate | 10.00 |
| Quaternium-18 hectorite | 1.20 |
| (Dimethicone/vinyl dimethicone) crosspolymer, | 5.00 |
| dimethicone | |
| Cyclomethicone | 25.00 |
| (Component B) | |
| Purified water | Remainder |
| 1,3-butylene glycol | 5.00 |
| Sodium citrate | 0.20 |
| Preservative | 0.30 |
| (Component C) | |
| Example A-1: cellulose acetate particles | 10.00 |
| Total | 100.0 |

### Example B-5

### Preparation of Lipstick Base Material

Components B shown in Table 8 were heated to 60° C., and mixed well. A component C was added thereto and the resultant mixture was dispersed well. Further, components A were added thereto and the resultant mixture was dissolved using a microwave oven, and then the mixture was mixed well. Then, the mixture was dissolved again by heating using the microwave oven, poured into a mold, and solidified under cooling. This solidified product was put in a lipstick container to prepare a lipstick base material. The tactile sensation of the resulting lipstick base material was evaluated by the method described above. The results are shown in Table 12.

TABLE 8

| Component | wt. % |
|---|---|
| (Component (A)) | |
| Ceresin | 4.27 |
| Microcrystalline wax | 1.55 |
| Deresinated candelilla wax | 5.03 |
| High boiling point paraffin | 3.07 |
| (Component B) | |
| Diisostearyl malate | 1.95 |
| Dipentaerythritol fatty acid ester | 6.22 |
| Adsorption refined lanolin | 2.52 |
| Liquid lanolin acetate | 13.34 |
| Glyceryl tri-2-ethylhexanoate | 19.02 |
| Liquid paraffin | 7.28 |
| Isotridecyl isononanoate | 3.21 |
| Diglyceryl triisostearate | 4.01 |
| Methylphenyl polysiloxane | 2.41 |
| Para-hydroxybenzoate | 0.07 |
| Diisostearyl malate | Remainder |
| Natural type vitamin E | 0.05 |
| (Component C) | |
| Example A-1: cellulose acetate particles | 10.00 |
| Total | 100.00 |

### Example B-6

### Preparation of Body Powder

Components A shown in Table 9 were mixed well using a mixer. The resulting body powder was filled into a container to prepare a body powder. The tactile sensation of the resulting body powder was evaluated by the method described above. The results are shown in Table 12.

TABLE 9

| Component | |
|---|---|
| (Component (A)) | wt. % |
| Talc | Remaining amount |
| Example A-1: cellulose acetate | 10.00 |
| particles | |
| Fragrance | Suitable amount |
| Total | 100.00 |

### Example B-7

### Preparation of Solid Face Powder

A solid face powder was prepared in accordance with the normal method for producing a cosmetic material. That is, talc and a color pigment shown in Table 10 were mixed with a blender. Also, cellulose acetate particles and all the powder portions including the color pigment and talc mixed previously with the blender were stirred using a Henschel mixer. Thereafter, an oil component (binder) was added thereto, the mixture was heated to 70° C., further stirred, and subjected to pulverizing, as necessary. The resultant product was compression-molded into a container of a gold dish to prepare a solid face powder. The tactile sensation of the resulting solid face powder was evaluated by the method described above. The results are shown in Table 12.

TABLE 10

| Component | wt. % |
|---|---|
| (Powder) | |
| Talc | 30.00 |
| Sericite | 20.00 |
| Kaolin | 15.00 |
| Titanium oxide | 5.00 |
| Zinc myristate | 5.00 |
| Magnesium carbonate | 5.00 |
| Color pigment | Suitable amount |
| Example A-1: cellulose acetate particles | 15.00 |
| (Binder) | |
| Tragacanth gum | 3.00 |
| Liquid paraffin | 2.00 |
| Others: a preservative, an antioxidant, | |
| and a fragrance are added in a | |
| suitable amount as necessary. | |
| Total | 100.00 |

### Example B-8

### Preparation of Solid Powder Eye Shadow

Powders shown in Table 11 were mixed well, a binder was uniformly dissolved and added to the powders. The resultant mixture was further mixed and then compression-molded to prepare a solid powder eye shadow. The tactile sensation of the resulting solid powder eye shadow was evaluated by the method described above. The results are shown in Table 12.

TABLE 11

| Component | wt. % |
|---|---|
| (Powder) | |
| Mica | 15.00 |
| Sericite | 5.00 |
| Pigment | 15.00 |
| Pearl pigment | 10.00 |
| Example A-1: cellulose acetate particles | 51.00 |
| (Binder) | |
| Methylpolysiloxane | 2.00 |
| (Other) | |
| Sorbitan sesquioleate | 2.00 |
| Others: an antioxidant, a fragrance, | |
| and a preservative are added in a | |
| suitable amount as necessary. | |
| Total | 100.00 |

### Example B-9

Liquid foundation was prepared in the same manner as in Example B-1 except that the cellulose acetate particles: Example A-1 in Table 4 were changed to the cellulose acetate particles: Example A-12. The tactile sensation of the resulting liquid foundation was evaluated by the method described above. The results are shown in Table 12.

### Example B-10

A sunscreen was prepared in the same manner as in Example B-12 except that the cellulose acetate particles: Example A-1 in Table 5 were changed to the cellulose acetate particles: Example A-2. The tactile sensation of the resulting sunscreen was evaluated by the method described above. The results are shown in Table 12.

### Example B-11

Powder foundation was prepared in the same manner as in Example B-12 except that the cellulose acetate particles: Example A-1 in Table 6 were changed to the cellulose acetate particles: Example A-3. The tactile sensation of the resulting powder foundation was evaluated by the method described above. The results are shown in Table 12.

### Example B-12

A makeup base was prepared in the same manner as in Example B-4 except that the cellulose acetate particles: Example A-1 in Table 7 were changed to the cellulose acetate particles: Example A-12. The tactile sensation of the resulting makeup base was evaluated by the method described above. The results are shown in Table 12.

### Example B-13

Liquid foundation was prepared in the same manner as in Example B-1 except that the cellulose acetate particles: Example A-1 in Table 4 were changed to the cellulose acetate particles: Example A-12. The tactile sensation of the resulting liquid foundation was evaluated by the method described above. The results are shown in Table 12.

### Example B-14

A sunscreen was prepared in the same manner as in Example B-12 except that the cellulose acetate particles: Example A-1 in Table 5 were changed to the cellulose acetate particles: Example A-2. The tactile sensation of the resulting sunscreen was evaluated by the method described above. The results are shown in Table 12.

### Example B-15

Liquid foundation was prepared in the same manner as in Example B-1 except that the cellulose acetate particles: Example A-1 in Table 4 were changed to the cellulose acetate particles: Example A-12. The tactile sensation of the resulting liquid foundation was evaluated by the method described above. The results are shown in Table 12.

### Example B-16

A sunscreen was prepared in the same manner as in Example B-12 except that the cellulose acetate particles: Example A-1 in Table 5 were changed to the cellulose acetate particles: Example A-2. The tactile sensation of the resulting sunscreen was evaluated by the method described above. The results are shown in Table 12.

### Example B-17

Liquid foundation was prepared in the same manner as in Example B-1 except that cyclopentasiloxane in Table 4 was replaced with a mixture prepared by mixing dodecane (PARAFOL 12-97 (Sasol)) and Cetiol Ultimate (undecane:tridecane=65 wt. %: 35 wt. %, available from BASF) in an identical weight ratio, isononyl isononanoate was replaced with a mixture prepared by mixing coco-caprylate (Cetiol C5 (BASF)), coco-caprylate/caprate (Cetiol C5C (BASF)), and dicaprylyl carbonate (Cetiol CC (BASF)) in an identical weight ratio, and phytosteryl macadamiate was replaced with camellia oil (Pure Tsubaki oil (Nikko Rica Corporation)). The tactile sensation of the resulting liquid foundation was evaluated by the method described above. The results are shown in Table 12.

### Example B-18

A sunscreen was prepared in the same manner as in Example B-2 except that isododecane in Table 5 was replaced with a mixture prepared by mixing dodecane (PARAFOL 12-97 (Sasol)) and Cetiol Ultimate (undecane:tridecane=65 wt. %: 35 wt. %, available from BASF) in an identical weight ratio, and diisopropyl sebacate was replaced with a mixture prepared by mixing coco-caprylate (Cetiol C5 (BASF)), coco-caprylate/caprate (Cetiol C5C (BASF)), and dicaprylyl carbonate (Cetiol CC (BASF)) in an identical weight ratio. The tactile sensation of the resulting sunscreen was evaluated by the method described above. The results are shown in Table 12.

### Example B-19

Powder foundation was prepared in the same manner as in Example B-3 except that dimethicone in Table 6 was replaced with a mixture prepared by mixing dodecane (PARAFOL 12-97 (Sasol)) and Cetiol Ultimate (undecane:tridecane=65 wt. %: 35 wt. %, available from BASF) in an identical weight ratio, and octyldodecyl oleate was replaced with a mixture prepared by mixing coco-caprylate (Cetiol C5 (BASF)), coco-caprylate/caprate (Cetiol C5C (BASF)), and dicaprylyl carbonate (Cetiol CC (BASF)) in an identical weight ratio. The tactile sensation of the resulting powder foundation was evaluated by the method described above. The results are shown in Table 12.

### Example B-20

A makeup base was prepared in the same manner as in Example B-4 except that cyclomethicone in Table 7 was replaced with a mixture prepared by mixing dodecane (PARAFOL 12-97 (Sasol)) and Cetiol Ultimate (undecane:tridecane=65 wt. %: 35 wt. %, available from BASF) in an identical weight ratio, and isononyl isononanoate was replaced with a mixture prepared by mixing coco-caprylate (Cetiol C5 (BASF)), coco-caprylate/caprate (Cetiol C5C (BASF)), and dicaprylyl carbonate (Cetiol CC (BASF)) in an identical weight ratio. The tactile sensation of the resulting makeup base was evaluated by the method described above. The results are shown in Table 12.

### Example B-21

Powder foundation was prepared in the same manner as in Example B-3 except that Mica Y-2300X in Table 6 was replaced with a mixture prepared by mixing mica (Mica Y-2300X (Yamaguchi Mica Co., Ltd.)), synthetic mica (PDM-10L (Topy Industries Limited), and (fluorinated/hydroxylated/oxidized)/(Mg/K/silicon) (Micro Mica MK-200K (Katakura & Co-op Agri Corporation) in an identical weight ratio, and sericite was replaced with a mixture prepared by mixing barium sulfate (plate-like barium sulfate H, available from Sakai Chemical Industry Co., ltd.) and boron nitride (SHP-6 (Mizushima Ferroalloy Co., Ltd.)) in an identical weight ratio, and talc was replaced with a mixture prepared by mixing cellulose (NP fiber W-06MG (Nippon Paper Industries Co., Ltd.)) and silica (Godd Ball E-16C (Suzukiyushi Industrial Corporation)) in an identical weight ratio. The tactile sensation of the resulting powder foundation was evaluated by the method described above. The results are shown in Table 12.

### Example B-22

A body powder was prepared in the same manner as in Example B-6 except that talc in Table 9 was replaced with a mixture prepared by mixing cellulose (NP fiber W-06MG (Nippon Paper Industries Co., Ltd.)) and silica (Godd Ball E-16C (Suzukiyushi Industrial Corporation)) in an identical weight ratio. The tactile sensation of the resulting body powder was evaluated by the method described above. The results are shown in Table 12.

### Example B-23

A solid powder eye shadow was prepared in the same manner as in Example B-8 except that Mica Y-2300X in Table 11 was replaced with a mixture prepared by mixing mica (Mica Y-2300X (Yamaguchi Mica Co., Ltd.)), synthetic mica (PDM-10L (Topy Industries Limited)), and (fluorinated/hydroxylated/oxidized)/(Mg/K/silicon) (Micro Mica MK-200K (Katakura & Co-op Agri Corporation) in an identical weight ratio, and sericite was replaced with a mixture prepared by mixing barium sulfate (plate-like barium sulfate H (Sakai Chemical Industry Co., Ltd.)) and boron nitride (SHP-6 (Mizushima Ferroalloy Co., Ltd.)) in an identical weight ratio. The tactile sensation of the resulting solid powder eye shadow was evaluated by the method described above. The results are shown in Table 12.

### Example B-24

A liquid foundation was prepared in the same manner as in Example B-1 except that BG in Table 4 was replaced with a mixture prepared by mixing glycerin and pentylene glycol (Diol PD (Kokyu Alcohol Kogyo Co., Ltd.)) in an identical weight ratio. The tactile sensation of the resulting liquid foundation was evaluated by the method described above. The results are shown in Table 12.

### Example B-25

A sunscreen was prepared in the same manner as in Example B-2 except that BG in Table 5 was replaced with a mixture prepared by mixing glycerin and pentylene glycol (Diol PD (Kokyu Alcohol Kogyo Co., Ltd.)) in an identical weight ratio. The tactile sensation of the resulting sunscreen was evaluated by the method described above. The results are shown in Table 12.

### Example B-26

A makeup base was prepared in the same manner as in Example B-4 except that 1,3-butylene glycol in Table 7 was replaced with a mixture prepared by mixing glycerin and pentylene glycol (Diol PD (Kokyu Alcohol Kogyo Co., Ltd.)) in an identical weight ratio. The tactile sensation of the resulting makeup base was evaluated by the method described above. The results are shown in Table 12.

### Comparative Examples B-1 to B-8

In Comparative Examples B-1 to B-8, liquid foundation, a sunscreen, powder foundation, a makeup base, a lipstick base, a body powder, a solid face powder, and a solid powder eye shadow were prepared in the same manner as in Examples B-1 to B-8 except that cellulose acetate particles in Example A-1 in Tables 4 to 11 were replaced with cellulose acetate particles in Comparative Example A-1. The tactile sensation of each of the products was evaluated by the method described above. The results are shown in Table 13.

TABLE 12

| | Example | Example | Example | Example | Example | Example | Example | Example |
|---|---|---|---|---|---|---|---|---|
| | B-1 | B-2 | B-3 | B-4 | B-5 | B-6 | B-7 | B-8 |
| Composition | Liquid | Sunscreen | Powder | Makeup | Lipstick | Body | Solid face | Solid |
| | foundation | | foundation | base | base | powder | powder | powder eye |
| | | | | | | | | shadow |
| Particles | Example | Example | Example | Example | Example | Example | Example | Example |
| | A-1 | A-1 | A-1 | A-1 | A-1 | A-1 | A-1 | A-1 |
| Tactile | 4.5 | 4.6 | 4.3 | 4.2 | 4.1 | 4.5 | 4.7 | 4.6 |
| sensation | | | | | | | | |
| | Example | Example | Example | Example | Example | Example | Example | Example |
| | B-9 | B-10 | B-11 | B-12 | B-13 | B-14 | B-15 | B-16 |
| Composition | Liquid | Sunscreen | Powder | Makeup | Liquid | Sunscreen | Liquid | Sunscreen |
| | foundation | | foundation | base | foundation | | foundation | |
| Particles | Example | Example | Example | Example | Example | Example | Example | Example |
| | A-12 | A-12 | A-12 | A-12 | A-12 | A-12 | A-12 | A-12 |
| Tactile | 4.1 | 4.2 | 4.1 | 4.3 | 4.3 | 4.4 | 4.1 | 4.1 |
| sensation | | | | | | | | |

| | Example | Example | Example | Example | Example | Example | Example | Example |
|---|---|---|---|---|---|---|---|---|
| | B-17 | B-18 | B-19 | B-20 | B-21 | B-22 | B-23 | B-24 |
| Composition | Liquid | Sunscreen | Powder | Makeup | Powder | Body | Solid | Liquid |
| | foundation | | foundation | base | foundation | powder | powder eye | foundation |
| | | | | | | | shadow | |
| Particles | Example | Example | Example | Example | Example | Example | Example | Example |
| | A-1 | A-1 | A-1 | A-1 | A-1 | A-1 | A-1 | A-1 |
| Tactile | 4.2 | 4.3 | 4.2 | 4.1 | 4.2 | 4.4 | 4.2 | 4.3 |
| sensation | | | | | | | | |

| | Example | Example |
|---|---|---|
| | B-25 | B-26 |
| Composition | Sunscreen | Makeup |
| | | base |
| Particles | Example | Example |
| | A-1 | A-1 |
| Tactile | 4.3 | 4.3 |
| sensation | | |

TABLE 13

| | Comparative | Comparative | Comparative | Comparative | Comparative | Comparative | Comparative | Comparative |
|---|---|---|---|---|---|---|---|---|
| | Example B-1 | Example B-2 | Example B-3 | Example B-4 | Example B-5 | Example B-6 | Example B-7 | Example B-8 |
| Composition | Liquid | Sunscreen | Powder | Makeup | Lipstick | Body | Solid face | Solid |
| | foundation | | foundation | base | base | powder | powder | powder eye |
| | | | | | | | | shadow |
| | Comparative | Comparative | Comparative | Comparative | Comparative | Comparative | Comparative | Comparative |
| Particles | Example A-1 | Example A-1 | Example A-1 | Example A-1 | Example A-1 | Example A-1 | Example A-1 | Example A-1 |
| Tactile | 3.1 | 2.9 | 3.2 | 3.1 | 3.3 | 3.2 | 3.2 | 3.1 |
| sensation | | | | | | | | |

As shown in Tables 12 and 13, all of the cosmetic compositions containing cellulose acetate particles of Examples B-1 to B-26 have a tactile sensation score of 4.0 or greater, particularly soft tactile sensation, and are excellent. Further, the cosmetic compositions contain cellulose acetate particles, and thus have excellent biodegradability.

### EMBODIMENTS

Embodiment 1-1. Cellulose acetate particles having an average particle size of 80 nm or more and 100 μm or less, a sphericity of 0.7 or more and 1.0 or less, and a surface smoothness of 80% or more and 100% or less, and the cellulose acetate having a total degree of acetyl substitution of 0.7 or more and 2.9 or less.
Embodiment 1-2. The cellulose acetate particles according to embodiment 1, wherein the total degree of acetyl substitution of the cellulose acetate is 2.0 or more and less than 2.6.
Embodiment 1-3. The cellulose acetate particles according to embodiment 1 or 2, wherein the cellulose acetate particles contain a plasticizer, and a content of the plasticizer is 2% by weight or more and 40% by weight or less based on a weight of the cellulose acetate particles.
Embodiment 1-4. The cellulose acetate particles according to embodiment 3, wherein the plasticizer is at least one or more selected from the group consisting of a citric acid-based plasticizer, a glycerin ester-based plasticizer, an adipic acid-based plasticizer, and a phthalic acid-based plasticizer.
Embodiment 1-5. A cosmetic composition containing the cellulose acetate particles according to any one of embodiments 1 to 4.
Embodiment 1-6. A method of producing cellulose acetate particles, the method comprising:

mixing cellulose acetate having a total degree of acetyl substitution of 0.7 or more and 2.9 or less with a plasticizer to obtain cellulose acetate impregnated with the plasticizer;

kneading the cellulose acetate impregnated with the plasticizer and a water-soluble polymer at 200° C. or more and 280° C. or less to obtain a dispersion having the cellulose acetate impregnated with the plasticizer as a dispersoid; and

removing the water-soluble polymer from the dispersion.

Embodiment 1-7. The method according to embodiment 6, wherein the mixing is performed by mixing the cellulose acetate and the plasticizer in a temperature range of 20° C. or more and less than 200° C. and then melt-kneading.
Embodiment 1-8. The method according to embodiment 6 or 7, wherein the plasticizer is at least one or more selected from the group consisting of a citric acid-based plasticizer, a glycerin ester-based plasticizer, an adipic acid-based plasticizer, and a phthalic acid-based plasticizer.
Embodiment 1-9. The method according to embodiment 6 or 7, wherein the plasticizer is at least one or more selected from the group consisting of triethyl citrate, acetyl triethyl citrate, acetyl tributyl citrate, triacetin, and diisononyl adipate.
Embodiment 1-10. The method according to embodiment 6 or 7, wherein the plasticizer is at least one or more selected from the group consisting of acetyl triethyl citrate, triacetin, diacetin, and diethyl phthalate.
Embodiment 1-11. The method according to any one of embodiments 6 to 10, wherein the water-soluble polymer is polyvinyl alcohol or thermoplastic starch.
Embodiment 2-1. Particles containing cellulose acetate, wherein the particles have an average particle size of not less than 80 nm and not greater than 100 μm, a sphericity of not less than 0.7 and not greater than 1.0, a degree of surface smoothness of not less than 80% and not greater than 100%, and a surface contact angle with water of not less than 100°; and a total degree of acetyl substitution of the cellulose acetate is not less than 0.7 and not greater than 2.9.
Embodiment 2-2. The particles according to embodiment 1, wherein the surface contact angle with water is not less than 120°.
Embodiment 2-3. The particles according to embodiment 1 or 2, wherein the total degree of acetyl substitution of the cellulose acetate is not less than 2.0 and less than 2.6.
Embodiment 2-4. The particles according to any one of embodiments 1 to 3, wherein the particles contain a plasticizer, and a content of the plasticizer is not greater than 1 wt. % relative to a weight of the particles.
Embodiment 2-5. The particles according to embodiment 4, wherein the plasticizer is at least one or more selected from the group consisting of a citrate-based plasticizer, a glycerin ester-based plasticizer, an adipate-based plasticizer, and a phthalate-based plasticizer.
Embodiment 2-6. The particles according to embodiment 5, wherein the glycerin ester-based plasticizer is triacetin.
Embodiment 2-7. A cosmetic composition containing the particles described in any one of embodiments 1 to 6.
Embodiment 2-8. A method for producing particles, the particles described in embodiment 1, the method comprising surface-treating cellulose acetate particles with a lipophilicity-imparting agent, wherein
the cellulose acetate particles have an average particle size of not less than 80 nm and not greater than 100 μm, a sphericity of not less than 0.7 and not greater than 1.0, and a degree of surface smoothness of not less than 80% and not greater than 100%; and
a total degree of acetyl substitution of the cellulose acetate is not less than 0.7 and not greater than 2.9.
Embodiment 2-9. The method for producing particles according to embodiment 8, wherein the lipophilicity-imparting agent comprises a silicone-based component.
Embodiment 2-10. The method for producing particles according to embodiment 9, the particles comprising cellulose acetate, wherein the surface treatment is a surface treatment by a wet treatment method.
Embodiment 3-1. An emulsifiable preparation including: one or more aqueous components selected from the group consisting of water and alcohol; an oily component; and microparticles of a polymer compound, in which the microparticles contain cellulose acetate as the polymer compound, and the microparticles has an average particle size of 2 to 10 μm.
Embodiment 3-2. The emulsifiable preparation according to embodiment 1, in which a content of the polymer compound contained in the microparticles is 60 wt. % or greater, 80 wt. % or greater, or 95 wt. % or greater relative to a total weight of the microparticles.
Embodiment 3-3. The emulsifiable preparation according to embodiment 1 or 2, in which a content of the cellulose acetate contained in the polymer compound is 40 wt. % or greater, 60 wt. % or greater, or 80 wt. % or greater, relative to a total weight of the polymer compound.
Embodiment 3-4. The emulsifiable preparation according to any one of embodiments 1-3, further including a polymer compound other than cellulose acetate (another polymer compound), in which the other polymer compound is at least one selected from the group consisting of biodegradable polymer compounds such as polylactic acid, polyglycolic acid, polyaspartic acid, polyvinyl alcohol, polyhydroxyalkanoate, modified polyethylene terephthalate, starch (glucose polymer), cellulose derivatives other than cellulose acetate, polybutylene succinate-based compounds, polycaprolactone, and gelatin; polysaccharides such as pullulan, gellan gum, xanthan gum, tamarind seed gum, locust bean gum, pectin, carrageenan, guar gum, gum arabic, dextran, dextrin, sodium chondroitin sulfate, sodium hyaluronate, and sodium alginate; polyvinylpyrrolidone, carboxyvinyl polymer, sodium polyacrylate, methacrylic acid copolymer, and polyethylene glycol.
Embodiment 3-5. The emulsifiable preparation according to any one of embodiments 1-4, in which the microparticles have an average particle size of 2 to 10 μm, or an upper limit of the average particle size is 8 μm or 7 μm, and a lower limit of the average particle size is 4 μm or 5 μm.
Embodiment 3-6. The emulsifiable preparation according to any one of embodiments 1-5, in which the cellulose acetate has a total degree of acetyl substitution of 0.7 or greater and 2.9 or less, 1.4 or greater and less than 2.6, or 2.0 or greater and less than 2.6.
Embodiment 3-7. The emulsifiable preparation according to any one of embodiments 1-6, in which the cellulose acetate is cellulose acetate impregnated with a plasticizer.
Embodiment 3-8. The emulsifiable preparation according to embodiment 7, in which the plasticizer is at least one selected from the group consisting of citrate-based plasticizers containing a citrate ester, such as triethyl citrate, acetyl triethyl citrate, and acetyl tributyl citrate; glycerin ester-based plasticizers containing a glycerin alkyl ester, such as triacetin, diacetin, and monoacetin; adipate-based plasticizers, such as diisononyl adipate; and phthalate-based plasticizers, such as ethyl phthalate and methyl phthalate.
Embodiment 3-9. The emulsifiable preparation according to embodiment 7 or 8, in which a blending amount of the plasticizer is more than 0 parts by weight and 40 parts by weight or less, 2 parts by weight or greater and 40 parts by weight or less, 10 parts by weight or greater and 30 parts by weight or less, or 15 parts by weight or greater and 20 parts by weight or less, relative to 100 parts by weight of the total amount of the cellulose acetate and the plasticizer.
Embodiment 3-10. The emulsifiable preparation according to any one of embodiments 1-9, in which the alcohol contains a polyhydric alcohol.
Embodiment 3-11. The emulsifiable preparation according to embodiment 10, in which the polyhydric alcohol is at least one selected from the group consisting of propylene glycol, dipropylene glycol, glycerin, diglycerin, low-molecular-weight (e.g., a weight-average molecular weight of 1000 or greater and 20000 or less) polyethylene glycol, 1,3-butylene glycol, and 1,2-pentanediol.
Embodiment 3-12. The emulsifiable preparation according to embodiment 10 or 11, in which an amount of the polyhydric alcohol is 20 wt. % or greater, 50 wt. % or greater, or 100 wt. % relative to a total amount of the alcohol.
Embodiment 3-13. The emulsifiable preparation according to any one of embodiments 1-12, further including a thickener.
Embodiment 3-14. The emulsifiable preparation according to any one of embodiments 1-13, further including a surfactant.
Embodiment 3-15. An aqueous cosmetic including the emulsifiable preparation described in any one of embodiments 1-14.
Embodiment 3-16. A food or beverage including the emulsifiable preparation described in any one of embodiments 1-14.
Embodiment 3-17. A pharmaceutical composition including the emulsifiable preparation described in any one of embodiments 1-14.
Embodiment 4-1. Cellulose acetate particles having:

an average particle size of 80 nm or greater and 100 μm or less, a sphericity of 0.7 or greater and 1.0 or less, and a relative specific surface area of 3.0 or greater and 20 or less; and a total degree of acetyl substitution of the cellulose acetate of 0.7 or greater and 3.0 or less.

Embodiment 4-2. The cellulose acetate particles according to embodiment 1, having a degree of surface smoothness of 10% or greater and 95% or less.
Embodiment 4-3. The cellulose acetate particles according to embodiment 1 or 2, having a bulk specific gravity of 0.2 or greater and 0.7 or less.
Embodiment 4-4. The cellulose acetate particles according to any one of embodiments 1 to 3, having an oil absorption using linseed oil of 60 ml or greater per 100 g of the cellulose acetate particles.
Embodiment 4-5. The cellulose acetate particles according to any one of embodiments 1 to 4, having the total degree of acetyl substitution of the cellulose acetate of 1.6 or greater and less than 2.9.
Embodiment 4-6. The cellulose acetate particles according to any one of embodiments 1 to 5, having the relative specific surface area of 10 or greater and 20 or less.
Embodiment 4-7. The cellulose acetate particles according to any one of embodiments 1 to 6, wherein the cellulose acetate particles contain a plasticizer, and a content of the plasticizer is 2 parts by weight or greater and 67 parts by weight or less relative to 100 parts by weight of the cellulose acetate.
Embodiment 4-8. The cellulose acetate particles according to embodiment 7, wherein the plasticizer contains at least one selected from the group consisting of a citrate-based plasticizer, a glycerin ester-based plasticizer, and a phthalate-based plasticizer.
Embodiment 4-9. A cosmetic composition comprising the cellulose acetate particles described in any one of embodiments 1 to 8.
Embodiment 4-10. A method for producing cellulose acetate particles, comprising:
mixing cellulose acetate having a total degree of acetyl substitution of 0.7 or greater and 3.0 or less, a plasticizer, a first thermoplastic polymer, and a second thermoplastic polymer, and forming a mixture of cellulose acetate containing the plasticizer, the first thermoplastic polymer, and the second thermoplastic polymer;
melt-kneading the mixture at 200° C. or higher and 280° C. or lower; and
removing the first thermoplastic polymer and the second thermoplastic polymer from the melt-kneaded mixture,
wherein, when SPa represents an SP value of the cellulose acetate, SPb represents an SP value of the first thermoplastic polymer, and SPc represents an SP value of the second thermoplastic polymer, SPa, SPb, and SPc satisfy the following relation:

0.1≤|SPc−SPa|/|SPb−SPa|≤0.9.

Embodiment 4-11. The method for producing cellulose acetate particles according to embodiment 10, wherein the plasticizer contains at least one selected from the group consisting of acetyl triethyl citrate and triacetin.
Embodiment 4-12. The method for producing cellulose acetate particles according to embodiment 10 or 11, wherein the first thermoplastic polymer contains at least one selected from the group consisting of polyvinyl alcohol and thermoplastic starch.
Embodiment 4-13. The method for producing cellulose acetate particles according to any one of embodiments 10 to 12, wherein the second thermoplastic polymer is polyethylene glycol.

1. A composition comprising cellulose acetate particles having an average particle size from 80 nm to 100 μm, a sphericity from 0.7 to 1.0, and a surface smoothness from 80% to 100% or less.

2. The composition according to claim 1, wherein the cellulose acetate particles have an average particle size from 80 nm to 80 μm.

3. The composition according to claim 1, wherein the cellulose acetate particles contain a plasticizer, and a content of the plasticizer is from 2% by weight to 40% by weight based on a weight of the cellulose acetate particles.

4. The composition according to claim 3, wherein the plasticizer is at least one or more selected from the group consisting of a citric acid-based plasticizer, a glycerin ester-based plasticizer, an adipic acid-based plasticizer, and a phthalic acid-based plasticizer.

5. The composition according to claim 1, wherein the cellulose acetate particles have a particle size variation coefficient from 0% to 60%.

6. The composition according to claim 1, wherein the cellulose acetate particles have a bulk density from 0.1 to 0.9 g/cm³.

7. The composition according to claim 1, wherein the average particle size is from 100 nm to 40 μm.

8. The composition according to claim 1, wherein the sphericity is from 0.9 to 1.0.

9. The composition according to claim 1, wherein the surface smoothness is from 90% to 100%.

10. A cosmetic composition containing the composition according to claim 1.

11. A method of producing the composition of claim 1, the method comprising:
a. mixing cellulose acetate with a plasticizer to obtain cellulose acetate impregnated with the plasticizer;
b. kneading the cellulose acetate impregnated with the plasticizer and a water-soluble polymer at 200° C. or more and 280° C. or less to obtain a dispersion having the cellulose acetate impregnated with the plasticizer as a dispersoid; and
c. removing the water-soluble polymer from the dispersion.

12. The method according to claim 6, wherein the mixing is performed by mixing the cellulose acetate and the plasticizer in a temperature range of 20° C. or more and less than 200° C. and then melt-kneading.

13. The method according to claim 6, wherein the plasticizer is at least one or more selected from the group consisting of a citric acid-based plasticizer, a glycerin ester-based plasticizer, an adipic acid-based plasticizer, and a phthalic acid-based plasticizer.

14. The method according to claim 6, wherein the plasticizer is at least one or more selected from the group consisting of triethyl citrate, acetyl triethyl citrate, acetyl tributyl citrate, triacetin, and diisononyl adipate.

15. The method according to claim 6, wherein the plasticizer is at least one or more selected from the group consisting of acetyl triethyl citrate, triacetin, diacetin, and diethyl phthalate.

16. The method according to claim 6, wherein the water-soluble polymer is polyvinyl alcohol or thermoplastic starch.
